(19) United States
(12) Patent Application Publication
(10) Pub. No.: US 2018/0171294 A1
(43) Pub. Date: Jun. 21, 2018

(54) IN VITRO ARTIFICIAL LYMPH NODE METHOD FOR SENSITIZATION AND EXPANSION OF T CELLS FOR THERAPY AND EPITOPE MAPPING

(71) Applicants: Brian J. Czerniecki, Tampa, FL (US); Lea Lowenfeld, Philadelphia, PA (US)

(72) Inventors: Brian J. Czerniecki, Tampa, FL (US); Lea Lowenfeld, Philadelphia, PA (US)

(73) Assignee: THE TRUSTEES OF THE UNIVERSITY OF PENNSYLVANIA, Philadelphia, PA (US)

(21) Appl. No.: 15/561,796

(22) PCT Filed: Feb. 28, 2016
(86) PCT No.: PCT/US0216/024540

§ 371 Date: Sep. 26, 2017

Related U.S. Application Data
(60) Provisional application No. 62/138,969, filed on Mar. 26, 2015.

(51) Int. Cl. C12N 5/0783; G01N 33/574; A61K 39/00

(52) U.S. Cl. CPC C12N 5/0636; G01N 33/574; A61K 2039/5154; A61K 2039/5158; A61K 2039/55533; A61K 39/0011

(57) ABSTRACT

A method of creating a microenvironment for culture expansion of T cells. The expanded T cells can be used for a variety of therapeutic and research purposes.

This application claims priority and benefit from U.S. Provisional Patent Application Ser. No. 62/138,969 filed on Mar. 26, 2015.

### FIELD

The present embodiments are directed to, in vitro artificial lymph node method for sensitization and expansion of T cells for therapy and epitope mapping and diagnostic monitoring methods, treatment methods and tools based thereon.

### BACKGROUND

The lifetime risk of breast cancer development is nearly one in eight. The erb-B2 oncogene (HER-2/neu) is a molecular driver that is overexpressed in a significant number of breast, ovarian, gastric esophageal, lung, pancreatic, prostate and other solid tumors. HER2 overexpression (“HER2^{pos}”), a molecular oncodriver in several tumor types including approximately 20-25% of breast cancers (Meric, F., et al., J. Am Coll. Surg. 194:488-501 (2002)), is associated with an aggressive clinical course, resistance to chemotherapy, and a poor overall prognosis in breast cancer (“BC”). See, Henson, E. S., Clin. Can. Res. 12:845-53 (2006) (“Henson, et al.”) and Wang, G. S., Mol. Med. Rep. 6:779-82 (2012). In incipient BC, HER2 overexpression is associated with enhanced invasiveness (Roses, R. E., et al., Cancer Epidemiol. Biomarkers & Prev. 18(5): 1386-9 (2009)), tumor cell migration (Wolf-Yadlin, A., et al., Molecular Systems Biology 2:54 (2006)), and the expression of proangiogenic factors (Wen, X. F., et al., Oncogene 25:6986-96 (2006)), suggesting a critical role for HER2 in promoting a tumorigenic environment. In a retrospective analysis of ductal carcinoma in situ (“DCIS”) patients, DCIS lesions overexpressing HER2 were over six times as likely to be associated with invasive breast cancer than were DCIS lesions without HER2 overexpression.

Although molecular targeting therapies targeting HER2, (i.e., HERCEPTIN®/trastuzumab), in combination with chemotherapy, have significantly improved survival in HER2^{pos} BC patients (Piccart-Gebhart., M. J., et al., N. Eng. J. Med. 353:1659-72 (2005)), a substantial proportion of patients become resistant to such therapies (Pohlmann, P. R., et al., Clin. Can. Res. 15:7479-91 (2009) (“Pohlman, et al.”)). Strategies to identify patient subgroups at high risk of treatment failure, as well as novel approaches to improve response rates to HER2-targeted therapies, are needed. Although molecular targeting therapies targeting HER2, i.e., trastuzumab, has resulted in tremendous positive clinical effect in this type of breast cancer, the almost universal resistance to the existing HER2 therapies in advanced disease states, plus disease relapse in a sizeable proportion of women who receive the targeted therapy prove the need for additional strategies targeting HER2. The promise of vaccines that activate the immune system against HER2 which seek to mitigate tumor progression and preventing recurrence while encouraging, is yet to be fully realized. There remains a need for additional tests and therapies to diagnose and treat HER2 breast cancer.

The role of systemic anti-HER2 CD4⁺ Th1 responses in HER2-driven breast tumorigenesis, have been recently elucidated. There has been identified a progressive loss of anti-HER2 CD4⁺ Th1 response across a tumorigenic continuum in HER2^{pos}-breast cancer, which appears to be HER2-specific and regulatory T-cell (T_{reg})-independent. Specifically, there is an inverse correlation of anti-HER2 CD4⁺ Th1 responses with HER2 expression and disease progression. Th1 reactivity profiles show a significant stepwise decline in anti-HER2 Th1 immunity across a continuum (HD (healthy donors)→BD (benign breast biopsy)→HER2^{neg}-DCIS (ductal carcinoma in situ)→HER2^{neg}-IBC (invasive breast cancer)→HER2^{pos}-DCIS→HER2^{pos}-IBC (invasive breast cancer) in HER2^{pos} breast tumorigenesis. See, Datta, J., et al., Oncolmmunology 4(10):e1027474. DOI:10.1080/2162402X.2015. U.S. Pat. No. 1,022,301 (2015) and U.S. Ser. No. 14/658,095 filed Mar. 13, 2015 (collectively hereinafter, “Datta, et al.”). The depressed anti-HER2 Th1 responses in HER2^{pos}-invasive breast cancer were differentially restored after HER2-pulsed type-1 polarized dendritic cell (“DC1”) vaccinations, but the depressed responses were not restored following HER2-targeted therapy with trastuzumab and chemotherapy (“T/C”) or by other standard therapies such as surgical resection or radiation. Id. The restored anti-HER2 Th1 responses also appear to be durable for at least about six months or longer.

The expansion of T lymphocyte subsets (CD4⁺ or CD8⁺) is an essential step to gain enough T cells to perform adoptive therapy, or to identify epitopes on target antigens for peptide-based vaccines. Expansion of T cells, in principle is a simple process. However, in practice, many technical problems exist including poor levels of expansion, premature activation-induced cell death (apoptosis), or loss of antigen specificity and/or function.

Part of the problem lies in the inability to replicate, in vitro, the environment inside the body where antigen-specific T cell expansion occurs, which is the lymph node. These are specialized tissues that contain a number of different cell types apart from T lymphocytes including antigen-presenting dendritic cells and stromal cells such as epithelial cells. Each of these cell types plays a different role (both currently defined and as yet incompletely characterized) by providing contact-dependent signals (surface receptors) and soluble signals (cytokines) important for T cell growth and maintenance of cell function.

There remains a need for new methods of treating cancer. In particular there is a need to have additional immunotherapeutic approaches for treating or preventing breast and other types of cancer. The present embodiments are so directed.

For a better understanding of exemplary embodiments, together with other and further features and advantages thereof, reference is made to the following description, taken in conjunction with the accompanying drawings, and the scope of the claimed embodiments will be pointed out in the appended claims.

### BRIEF SUMMARY

In one broad aspect, there is provided a method of expanding a T cell population which comprises at least one T cell obtained from a blood sample from a subject who has been vaccinated against an antigen, comprising the step of: contacting the T cell with one or more of a dendritic cell (“DC”) or a precursor thereof, at least two cytokines, and a T cell growth factor.

In another aspect, the blood sample contains at least one T cell of the population specific for the vaccine antigen and at least one DC precursor.

In another aspect, the DC precursor is pulsed with the antigen and activated to an antigen-specific type I dendritic cell (“DC1”) and then co-cultured with the T cell to generate an antigen-specific DC1.

In another aspect, the at least two cytokines comprises interleukin-7 (“IL-7”) and interleukin-15 (“IL-15”).

In another aspect, the T cell growth factor comprises interleukin-2 (“IL-2”).

In another aspect, the method further comprises the steps of:
a) co-culturing the T cell from the patient sample with the antigen-specific T cell autologous type I dendritic cell (DC1) in vitro;
b) contacting the cell from step a) with IL-7 and IL-5 to generate a stimulated antigen-specific T cell, and
c) subsequently contacting the stimulated antigen specific T cell with IL-2, thereby generating an expanded antigen specific T cell population that maintains antigen specificity and cellular function.

In another aspect, the methods further comprises repeating steps a) through c) from one to at least three additional times to generate further expanded antigen-specific T cell populations.

In another aspect, the T cell is CD4⁺.

In another aspect, the antigen is HER2.

In another broad aspect, there is a method of expanding a CD4⁺ T cell population which comprises at least one CD4⁺ T cell obtained from a blood sample from a breast cancer patient who has been vaccinated against HER2, comprising the step of: contacting the CD4⁺ T cell with one or more of a dendritic cell (“DC”) or a precursor thereof, at least two cytokines, and a T cell growth factor.

In another aspect, at least one DC precursor in the sample is pulsed with at least one HER2 MHC class II peptide and is contacted with the CD4⁺ T cell.

In another aspect, the at least two cytokines comprises interleukin-7 (“IL-7”) and interleukin-15 (“IL-15”).

In another aspect, the T cell growth factor comprises interleukin-2 (“IL-2”).

In another aspect, the method comprises:
a) co-culturing the T cell with the HER2-pulsed DC1;
b) contacting the cell from step a) with IL-7 and IL-15 to generate a stimulated antigen-specific T cell, and
c) subsequently contacting the stimulated antigen specific T cell with IL-2, thereby generating an expanded antigen specific T cell population that maintains antigen specificity and cellular function.

In another aspect, the method further comprises repeating steps a) through c) from one to at least three additional times to generate further expanded antigen-specific T cell populations.

In another aspect, the sample is pulsed with HER2 MHC class II peptides, comprising:

Peptide 42-56:
(SEQ ID NO: 1)
HLDMLRHLYQGCQVV;
Peptide 98-114:
(SEQ ID NO: 2)
RLRIVTRGTQLFEDNYAL;
Peptide 328-345:
(SEQ ID NO: 3)
TQRCEKCSKPCARVCYGL;
Peptide 776-790:
(SEQ ID NO: 4)
GVGSPYVSRLLGICL;
Peptide 927-941:
(SEQ ID NO: 5)
PAREIPDLLEKGERL;
and
Peptide 1166-1180:
(SEQ ID NO: 6)
TLERPKTLSPGKNGV.

For a better understanding of exemplary embodiments, together with other features and advantages thereof, reference is made to the following description, taken in conjunction with the accompanying drawings, and the scope of the claimed embodiments will be pointed out in the appended claims.

### BRIEF DESCRIPTION OF THE DRAWINGS

The following detailed description of preferred embodiments will be better understood when read in conjunction with the appended drawings. For the purpose of illustrating the embodiments, there are shown in the drawings embodiments which are presently preferred. It should be understood, however, that the preferred embodiments are not limited to the precise arrangements and instrumentalities of the embodiments shown in the drawings.

FIG. 1 shows anti-HER2 Th1 response repertoire of four HER2⁺ IBC patients with residual disease following neoadjuvant therapy who received adjuvant HER2-pulsed DC1 vaccines. Each patient is depicted in a different color and shows number of reactive peptides (n) (also referred to as “response repertoire”) pre-vaccine, 3-months-post vaccine, and 6-months post vaccine. Mean response repertoire increased from 0.5±1 peptides pre-vaccination to 3.25±0.96 peptides at 3 months post-vaccination (p=0.01) and 4±0.8 peptides at 6 months post-vaccination (p=0.01).

FIG. 2 shows anti HER2 Th1 cumulative response of four HER2⁺ IBC patients with residual disease following neoadjuvant therapy who received adjuvant HER2-pulsed DC1 vaccines. Each patient is depicted in a different color and shows cumulative response (SFC/10⁶ cells) pre-vaccine, 3-months-post vaccine, and 6-months post vaccine. Patient mean cumulative response improved from 36.5±38.3 SFC/10⁶ pre-vaccination to 151.0±60.0 SFC/10⁶ at 3 months post vaccination (p=0.04) and 198.4±39.7 SFC/10⁶ at 6 months post vaccination (p=0.02).

FIG. 3 and FIG. 4 show a direct comparison between CD4⁺ T cells co-cultured with HER2-specific DC1's from patients vaccinated with HER2 peptide-pulsed DC1 vaccines stimulated with IL-2 versus those stimulated with IL-2/7/15 for two different patients, respectively. Immature DC's (“iDC's”) from the respective patients were pulsed with the following MHC class II peptides: peptide 42-56 (SEQ ID NO: 1), peptide 98-114 (SEQ ID NO: 2), peptide 328-345 (SEQ ID NO: 3), and peptide 776-790 (SEQ ID NO: 4) and matured to DC1's. The resulting HER2-pulsed DC1's were then co-cultured with CD4⁺ T cells and stimulated with IL-2 alone or with IL-2/7/15 as indicated. The red outline boxes indicate the specific peptide and stimulation protocol for which specificity is shown (greater than 2:1 ratio of specific antigen:control antigen IFN-γ production). For each set of peptide/stimulation protocol: “Control antigen” shows non-specific iDC's co-cultured with control antigen; “Specific antigen” represents anti-HER2 CD4⁺ T cells co-cultured with iDC's that were pulsed with HER2 antigen/peptide; and “Tcell” represents anti-HER2 CD4⁺ T cells in culture medium. Graphs showing fold expansion (defined as number of T cells post expansion/number of T cells pre expansion) are shown at right, respectively. Specificity was measured by antigen-specific IFN-γ production by ELISA.

FIG. 5 and FIG. 6 show specific responses followed by non-specific immune responses: FIG. 5 shows a specific response following a first stimulation/expansion with HER2-specific DC1's and FIG. 6 shows the subsequent loss of that specific response after the second stimulation/expansion with non-specific anti CD3/CD28. The first stimulation of CD4⁺ T cells with HER2-specific DC1s resulted in multiple specific immune responses as shown by red outline boxes in FIG. 5. FIG. 6 shows the second stimulation of the HER2-specific CD4⁺ T cells with a non-specific anti-CD3/CD28 stimulus resulted in a four-fold expansion (side graph), but with a loss of specificity in three fourths of the peptide groups. iDC's from patients were pulsed with the following MHC class II peptides: peptide 42-56 (SEQ ID NO: 1), peptide 98-114 (SEQ ID NO: 2), peptide 328-345 (SEQ ID NO: 3), and peptide 776-790 (SEQ ID NO: 4) and matured to DC1's. The resulting HER2-pulsed DC1 's were then co-cultured with CD4⁺ T cells and stimulated with IL-2 alone or with IL-2/7/15 as indicated.

FIG. 7 and FIG. 8 show non-specific immune response followed by specific immune responses: FIG. 7 shows non-specific expansion of CD4⁺ T cells. FIG. 8 shows failure to obtain specificity following subsequent stimulation with HER2-specific DC1's. The first stimulation of CD4⁺ T cells with non-specific anti-CD3/CD28 resulted in a 3.8 fold expansion (FIG. 7). The second stimulation of the non-specific CD4⁺ T cells with HER2-specific DC1's failed to result in a specific immune response (FIG. 8). iDC's from patients were pulsed with the following MHC class II peptides: peptide 42-56 (SEQ ID NO: 1), peptide 98-114 (SEQ ID NO: 2), peptide 328-345 (SEQ ID NO: 3), and peptide 776-790 (SEQ ID NO: 4) and matured to DC1's. The resulting HER2-pulsed DC1's were then co-cultured with CD4⁺ T cells and stimulated with IL-2 alone or with IL-2/7/15 as indicated.

FIGS. 9A and 9B show in vitro primary/first expansion of HER2-specific Th1 cells comparing CD4⁺ T cells co-cultured with HER2-specific DC1's expanded with IL-2 versus those expanded with IL-2/7/15. Immature DC's (“iDC's”) were pulsed with the following MHC class II peptides: peptide 42-56 (SEQ ID NO: 1), peptide 98-114 (SEQ ID NO: 2), peptide 776-790 (SEQ ID NO: 4), and peptide 927-941 (SEQ ID NO: 5), and matured to DC1's. The resulting HER2-pulsed DC1's were then co-cultured with CD4⁺ T cells and stimulated with IL-2 alone or with IL-2/7/15 as indicated. The red outline boxes (FIG. 9B) indicate the specific peptide and stimulation protocol for which specificity is shown (greater than 2:1 ratio of specific antigen:control antigen IFN-γ production). For each set of peptide/stimulation protocol: “Control Antigen” shows non-specific iDC's co-cultured with control antigen; “Specific Antigen” represents anti-HER2 CD4⁺ T cells co-cultured with iDC's that were pulsed with HER2 antigen/peptide; and “T cells” represents anti-HER2 CD4⁺ T cells in culture medium. FIG. 9A shows mean fold expansion (defined as number of T cells post expansion/number of T cells pre expansion) of Th1 cells was significantly better when stimulated with IL-2, IL-7, and IL-15 than with IL-2 alone (2.6±0.75 vs 1.0±0.12; p=0.001). FIG. 9B shows specificity for the various peptide/expansion protocols as measured by antigen-specific IFN-γ production by ELISA. Both stimulation with IL-2, IL-7, and IL-15 and with IL-2 alone resulted in a specific Th1 response in the same HER2 peptide 776-790.

FIGS. 10A and 10B show in vitro secondary/second expansion of HER2-pulsed DC1's versus anti-CD3/CD28. Re-stimulation of Th1 cells with HER2-peptide pulsed DC1s and anti-CD3/CD28 each resulted in a similar fold expansion (3.9±1.0 vs. 4.3±2.0 p=0.7) (FIG. 10A). However, FIG. 10B shows stimulation of the Th1 cells with HER2-specific DC1s enhanced the specific Th1 response; whereas non-specific stimulation with anti-CD3/CD28 resulted in an overall loss of HER2-peptide specificity. The following MHC class II peptides: peptide 42-56 (SEQ ID NO: 1), peptide 98-114 (SEQ ID NO: 2), peptide 776-790 (SEQ ID NO: 4), and peptide 927-941 (SEQ ID NO: 5) were used. The red outline boxes (FIG. 10B) indicate the specific peptide and stimulation protocol for which specificity is shown (greater than 2:1 ratio of specific antigen:control antigen IFN-γ production) (i.e., DC restimulation of peptide 42-56- and peptide 776-790-specific Th1 cells. For each set of peptide/stimulation protocol: “Control Antigen” shows non-specific iDC's co-cultured with control antigen; “Specific Antigen” represents anti-HER2 CD4⁺ T cells co-cultured with iDC's that were pulsed with HER2 antigen/peptide; and “T cells” represents anti-HER2 CD4⁺ T cells in culture medium.

FIGS. 11A and 11B show tertiary/third expansion of the Th1 cells with HER2-pulsed DC1's (peptide 42-56 (SEQ ID NO: 1), peptide 98-114 (SEQ ID NO: 2), peptide 776-790 (SEQ ID NO: 4), and peptide 927-941 (SEQ ID NO: 5) were used). Following a third stimulation with indicated HER2-specific DC1s, both mean fold expansion (4.32±0.5, 43.7-fold cumulative expansion (FIG. 11A) and antigen specificity (FIG. 11B) increased again, specifically all four peptides show specificity and increased IFN-γ production.

FIGS. 12-15 show sequential results of repeated in vitro stimulation (4 times) of HER2-specific CD4⁺ Th1 cells with IL-2/7/15. For all FIGS. 12-15 the respective left panels show peptide specificity by IFN-γ production (“Tet” is a tetanus patient control); respective right panels show fold expansion for the specific HER2-peptides used. In FIG. 12 two additional MHC-class II peptides were used to pulse iDC's: peptide 927-941 (SEQ ID NO: 5); and peptide 1166-1180 (SEQ ID NO: 6) in addition to the other four used in above figures. However, as seen in the fold expansion results (FIG. 12, right panel), peptide 328-345-specific and peptide 1166-1180-specific Th1 cells did not produce enough cells for further expansion, thus only HER2 Th1 cells specific to the remaining four peptides were so used. Sequentially, FIG. 12 for the first stimulation shows specificity only for peptide 776-790-specific Th1 cells; FIG. 13 for the second stimulation shows an increase, specificity for peptide 42-56- and peptide 776-790-specific Th1 cells; FIG. 14 for the third expansion shows specificity for all four peptides, and FIG. 15 for the fourth expansion shows loss of specificity for one of the peptides (peptide 927-941) leaving three remaining HER2-specific peptides.

FIG. 16 shows cumulative fold expansion of the four expansions shown in FIGS. 12-15 for all the HER2-specific Th1 cells, with the last bar of each group (dots) showing cumulative fold expansion.

### DETAILED DESCRIPTION

It is to be understood that the figures, images and descriptions of the present embodiments have been simplified to illustrate elements that are relevant for a clear understanding, while eliminating, for the purposes of clarity, many other elements which may be found in the present embodiments. Those of ordinary skill in the pertinent art will recognize that other elements are desirable and/or required in order to implement the present embodiments. However, because such elements are well known in the art, and because such elements do not facilitate a better understanding of the present embodiments, a discussion of such elements is not provided herein.

Reference throughout this specification of “one embodiment” or “an embodiment” or the like means that a particular feature, structure, or characteristic described in connection with the embodiment is included in at least one embodiment. Thus appearances of the phrases “in one embodiment” or “in an embodiment” or the like in various places throughout this specification are not necessarily all referring to the same embodiment.

In addition, for the purpose of promoting an understanding of the principles of the present disclosure, reference will now be made to the embodiments shown and described herein, and specific language will be used to describe the same. It will, nevertheless, be understood that no limitation of the scope of the disclosure is thereby intended; any alterations and further modifications of the described or illustrated embodiments and any further applications of the principles of the disclosure as illustrated herein are contemplated as would normally occur to one skilled in the art to which the disclosure relates. All limitations of scope should be determined in accordance with and as expressed in the eventual claims of one or more issued patents.

### Definitions

Unless defined otherwise, all technical and scientific terms used herein have the same meaning as commonly understood by one of ordinary skill in the art to which the inventive subject matter of this disclosure belongs. Although any methods and materials similar or equivalent to those described herein can be used in the practice or testing of the present embodiments, the preferred methods and materials are described.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an embodiment” means one embodiment or more than one embodiment.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

“Adjuvant therapy” for breast cancer as used herein refers to any treatment given after primary therapy (i.e., surgery) to increase the chance of long-term survival. “Neoadjuvant therapy” is treatment given before primary therapy.

The term “antigen” or “ag” as used herein is defined as a molecule that provokes an immune response. This immune response may involve either antibody production, or the activation of specific immunologically-competent cells, or both. One of ordinary skill in the art will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present embodiments include, but are not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated or synthesized or can be derived from a biological sample. Such a biological sample can include, but is not limited to a tissue sample, a tumor sample, a cell or a biological fluid.

An “antigen presenting cell” or “APC” is a cell that is capable of activating T cells, and includes, but is not limited to, monocytes/macrophages, B cells and dendritic cells (“DCs”).

“Antigen-pulsed APC” or an “antigen-loaded APC” includes an APC which has been exposed to an antigen and activated by the antigen. For example, an APC may become Ag-loaded in vitro, e.g., during culture in the presence of an antigen. An APC may also be loaded in vivo by exposure to an antigen. An “antigen-loaded APC” is traditionally prepared in one of two ways: (1) small peptide fragments, known as antigenic peptides, are “pulsed” directly onto the outside of the APCs; or (2) the APC is incubated with whole proteins or protein particles which are then ingested by the APC. These proteins are digested into small peptide fragments by the APC and are eventually transported to and presented on the APC surface. In addition, an antigen-loaded APC can also be generated by introducing a polynucleotide encoding an antigen into the cell.

“Anti-HER2 response” is the immune response specifically against HER2 protein.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of binding peptides, polynucleotides, cells and antibodies in prevention of the occurrence of tumor in the first place.

“Apoptosis” is the process of programmed cell death. Caspase-3 is a frequently activated death protease.

As used herein, the term “autologous” refers to any material derived from the same individual to which it is later to be introduced.

The term “B cell” as used herein is defined as a cell derived from the bone marrow and/or spleen. B cells can develop into plasma cells which produce antibodies.

“Binding peptides.” See, “HER2 binding peptides.”

The term “cancer” as used herein is defined as a hyperproliferation of cells whose unique trait—loss of normal control—results in unregulated growth, lack of differentiation, local tissue invasion, and/or metastasis. Examples include but are not limited to, breast cancer, prostate cancer, ovarian cancer, cervical cancer, skin cancer, bladder cancer, esophageal cancer, pancreatic cancer, colorectal cancer, gastric cancer, renal cancer, liver cancer, brain cancer, lymphoma, leukemia, lung cancer, germ-cell tumors, and the like.

“CD4⁺ Th1 cells,” “Th1 cells,” “CD4⁺ T-helper type 1 cells,” “CD4⁺ T cells,” and the like are defined as a subtype of T-helper cells that express the surface protein CD4 and produce high levels of the cytokine IFN-γ. See also, “T-helper cells.”

“Cumulative response” means the combined immune response of a patient group expressed as the total sum of reactive spots (spot-forming cells “SFC” per 10⁶ cells from IFN-γ ELISPOT analysis) from all 6 MHC class II binding peptides from a given patient group.

“DC vaccination,” “DC immunization,” “DC1 immunization,” and the like refer to a strategy using autologous dendritic cells to harness the immune system to recognize specific molecules and mount specific responses against them.

The term “dendritic cell” or “DC” is an antigen presenting cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. DCs have a characteristic morphology with thin sheets (lamellipodia) extending in multiple directions away from the dendritic cell body. Typically, dendritic cells express high levels of MHC and costimulatory (e.g., B7-1 and B7-2) molecules. Dendritic cells can induce antigen specific differentiation of T cells in vitro, and are able to initiate primary T cell responses in vitro and in vivo. In the context of vaccine production, an “activated DC” is a DC that has been exposed to a Toll-like receptor agonist such as lipopolysaccharide “LPS.” An activated DC may or may not be loaded with an antigen. See also, “mature DC.”

“DC-1 polarized dendritic cells,” “DC1s” and “type-1 polarized DCs” refer to mature DCs that secrete Th1-driving cytokines, such as IL-12, IL-18, and IL-23. DC s are fully capable of promoting cell-mediated immunity. DC1s are pulsed with HER2 MHC class II-binding peptides in preferred embodiments herein.

“HER2” is a member of the human epidermal growth factor receptor (“EGFR”) family. HER2 is overexpressed in approximately 20-25% of human breast cancer and is expressed in many other cancers.

“HER2 binding peptides,” “HER2 MHC class II binding peptides,” “binding peptides,” “peptide antigens,” “HER2 peptides,” “immunogenic MHC class II binding peptides,” “antigen binding peptides,” “HER2 epitopes,” “reactive peptides,” and the like as used herein refer to MHC Class II peptides derived from or based on the sequence of the HER2/neu protein, a target found on approximately 20-25% of all human breast cancers and their equivalents. HER2 extracellular domain “ECD” refers to a domain of HER2 that is outside of a cell, either anchored to a cell membrane, or in circulation, including fragments thereof. HER2 intracellular domain “ICD” refers to a domain of the HER2/neu protein within the cytoplasm of a cell. According to a preferred embodiment HER2 epitopes or otherwise binding peptides comprise 6 HER2 binding peptides which include 3 HER2 ECD peptides and 3 HER2 ICD peptides.

Preferred HER2 ECD peptides comprise:

Peptide 42-56:
(SEQ ID NO: 1)
HLDMLRHLYQGCQVV;
Peptide 98-114:
(SEQ ID NO: 2)
RLRIVRGTQLFEDNYAL;
and
Peptide 328-345:
(SEQ ID NO: 3)
TQRCEKCSKPCARVCYGL;

Preferred HER2 ICD peptides comprise:

Peptide 776-790:
(SEQ ID NO: 4)
GVGSPYVSRLLGICL;
Peptide 927-941:
(SEQ ID NO: 5)
PAREIPDLLEKGERL;
and
Peptide 1166-1180:
(SEQ ID NO: 6)
TLERPKTLSPGKNGV.

“HER2^{pos}” is the classification or molecular subtype of a type of breast cancer as well as numerous other types of cancer. HER2 positivity is currently defined by gene amplification by FISH (fluorescent in situ hybridization) assay and 2+ or 3+ on intensity of pathological staining.

“HER2^{neg}” is defined by the lack of gene amplification by FISH, and can encompass a range of pathologic staining from 0 to 2+ in most cases.

Interleukin 2 (“IL-2” or “IL2”) is an interleukin, a type of cytokine signaling molecule in the immune system. IL-2 is the principal T cell growth and proliferation factor.

Interleukin 7 (“IL-7” or “IL7”) is a hematopoietic growth factor produced by stromal epithelial cells in lymph nodes. IL-7 is essential for lymphocyte proliferation and survival.

Interleukin 15 (“IL-15” or “IL15”)) is a T cell growth activation and survival factor. IL-15 is produced by fibroblasts, dendritic cells and macrophages.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “major histocompatibility complex” or “MHC” as used herein is defined as a specific cluster of genes, many of which encode evolutionary related surface proteins involved in antigen presentation, which are among the most important determinants of histocompatibility. Class I MHC, or MHC class I, function mainly in antigen presentation to CD8 T lymphocytes. Class II MHC, or MHC class II, function mainly in antigen presentation to CD4⁺ T lymphocytes (T-helper cells).

“Mature DC” as used herein means a dendritic cell that expresses molecules, including high levels of MHC class II, CD80 (B7.1) and CD86 (B7.2) molecules. In contrast, immature DCs (“iDCs”) express low levels of MHC class II, CD80 (B7.1) and CD86 (B7.2) molecules, yet can still take up an antigen. “Mature DC” also refers to an antigen presenting cell existing in vivo, in vitro, ex vivo, or in a host or subject that may also be DC1-polarized (i.e., fully capable of promoting cell-mediated immunity.)

“Metrics” of CD4⁺ Th1 responses (or “Th1 responses”) are defined for each subject group analyzed for anti-HER2 CD4⁺ Th1 immune response: (a) overall anti-HER2 responsivity (expressed as percent of subjects responding to ≥1 reactive peptide), (b) response repertoire (expressed as mean number of reactive peptides (n) recognized by each subject group); and (c) cumulative response (expressed as total sum of reactive spots (spot-forming cells “SFC” per 10⁶ cells from IFN-γ ELISPOT analysis) from 6 MHC Class II binding peptides from each subject group).

“Non-equivocal HER2^{neg} is defined as non-gene amplified and 0 or 1+ on pathologic staining. “Equivocal HER2^{neg}” is defined as non-gene amplified but 2+ on pathologic staining.

“Responsivity” or “anti-HER2 responsivity” are used interchangeably herein to mean the percentage of subjects responding to at least 1 of 6 binding peptides.

“Response repertoire” is defined as the mean number (“n”) of reactive peptides recognized by each subject group.

“Sample” or “biological sample” as used herein means a biological material from a subject, including but is not limited to blood, organ, tissue, exosome, plasma, saliva, urine and other body fluid. A sample can be any source of material obtained from a subject.

The terms “subject,” “patient,” “individual,” and the like are used interchangeably herein, and refer to any animal, or cells thereof, whether in vitro or in situ, amenable to the methods described herein. In certain non-limiting embodiments, the patient, subject or individual is a human.

The term “targeted therapies” as used herein refers to cancer treatments that use drugs or other substances that interfere with specific target molecules involved in cancer cell growth usually while doing little damage to normal cells to achieve an anti-tumor effect. Traditional cytotoxic chemotherapy drugs, by contrast, act against all actively dividing cells. In breast cancer treatment monoclonal antibodies, specifically trastuzumab/HERCEPTIN® targets the HER2/neu receptor.

“T/C” is defined as trastuzumab and chemotherapy. This refers to patients that receive both trastuzumab and chemotherapy before/after surgery for breast cancer.

The terms “T cell” or “T-cell” as used herein are defined as a thymus-derived cell that participates in a variety of cell-mediated immune reactions.

The terms “T-helper cells,” “helper T cells,” “Th cells,” and the like are used herein with reference to cells indicates a sub-group of lymphocytes (a type of white blood cell or leukocyte) including different cell types identifiable by a skilled person in the art. In particular, T-helper cells are effector T cells whose primary function is to promote the activation and functions of other B and T lymphocytes and/or macrophages. Helper T cells differentiate into two major subtypes of cells known as “Th1” or “Type 1” and “Th2” or “Type 2” phenotypes. These Th cells secrete cytokines, proteins, or peptides that stimulate or interact with other leukocytes. “Th1 cell,” “CD4⁺ Th1 cell,” “CD4⁺ T-helper type1 cell,” “CD4⁺ T cell” and the like as used herein refer to a mature T-cell that has expressed the surface glycoprotein CD4. CD4⁺ T-helper cells become activated when they are presented with peptide antigens by MHC class II molecules which are expressed on the surface of antigen-presenting peptides (“APCs”) such as dendritic cells. Upon activation of a CD4⁺ T helper cell by the MHC-antigen complex, it secretes high levels of cytokines such as interferon-γ (“IFN-γ”). Such cells are thought to be highly effective against certain disease-causing microbes that live inside host cells, and are critical in antitumor response in human cancer.

The term “cytotoxic T cell” or “CD8⁺ T cell or “killer T cell” is a T lymphocyte that kills target cells such as cancer cells, cells that are infected, or cells that are damaged in other ways.

“Treg” “T_{reg}” and “regulatory T-cells” are used herein to refer to cells which are the policemen of the immune system, and which act to regulate the anti-cancer activities of the immune system. They are increased in some cancers, and are mediators in resistance to immunotherapy in these cancer types.

“Therapeutically effective amount” or “effective amount” are used interchangeably herein, and refer to an amount of a compound, formulation, material, or composition, as described herein, that when administered to a patient, is effective to achieve a particular biological result. The amount of a compound, formulation, material, or composition described herein, which constitutes a “therapeutically effective amount” will vary depending on the compound, formulation, material, or composition, the disease state and its severity, the age of the patient to be treated, and the like. The therapeutically effective amount can be determined routinely by one of ordinary skill in the art having regard to his/her own knowledge and to this disclosure.

The terms “treat,” “treating,” and “treatment,” refer to therapeutic or preventative measures described herein. The methods of “treatment” employ administration to a subject, in need of such treatment, a composition or method of the present embodiments, for example, a subject afflicted with a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

The term “vaccine” as used herein is defined as a material used to provoke an immune response after administration of the material to an animal, preferably a mammal, and more preferably a human. Upon introduction into a subject, the vaccine is able to provoke an immune response including, but not limited to, the production of antibodies, cytokines and/or other cellular responses.

Ranges: throughout this disclosure, various aspects of the embodiments can be presented in a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Reference will now be made in detail to several embodiments, examples of which are also illustrated in the accompanying drawings, photographs, and/or illustrations.

### Description

The present embodiments relate to HER2⁺ invasive breast cancer (“IBC”) patients with residual disease following neoadjuvant chemotherapy having an anti-HER2 Type 1 T helper (Th1) cell immune deficit and a significant risk of recurrent disease. It was shown in Datta, et al. that anti-HER2 CD4⁺ T cell responses incrementally decrease along the breast cancer continuum—a robust response in healthy donors and patients with benign disease, a depressed response in patients with HER2⁺ ductal carcinoma in situ, and a nearly absent response in patients with HER2⁺ IBC. Herein is explored the role of (A) adjuvant type 1-polarized dendritic cell (“DC1”) vaccination and (B) methods of expanding antigen-specific t-cells for adoptive T-cell transfer in restoring anti-HER2 Th1 immunity.

The present embodiments also relate to a method of creating a microenvironment in vitro for culture expansion of antigen-specific CD4⁺ or CD8⁺ T cells. The expanded antigen-specific T cells can be used for a variety of therapeutic and research purposes, for example adoptive T cell therapy for cancer or infectious disease such as chronic viral infections other conditions and/or for the identification of epitopes on target antigens to foster the production of peptide-based vaccines.

One of the present embodiments, uses autologous type I dendritic cells (“DC1s”) in combination with a protein or peptide antigen to stimulate T cells in vitro. After stimulation, at least two soluble factors (e.g., cytokines) are added to the T cells. In some instances, the at least two soluble factors are Interleukin-7 (“IL-7”) and Interleukin-15 (“IL-15”). Following the addition of the soluble factors to the T cells, a T cell growth factor is added. In some instances, the T cell growth factor is Interleukin-2 (“IL-2”). The soluble factors, in addition to those naturally produced by the DC1s, support the proliferation and acquisition/maintenance of T cell function. This process of stimulation can be repeated in weekly cycles until T cells are of sufficient numbers for therapy or epitope scanning/mapping. In certain embodiments, the T cells are expanded to a level necessary for adoptive therapy and epitope mapping studies while maintaining antigen specificity and cellular function.

In Vivo Th1 Response to HER-2-Pulsed DC1 Vaccination: Restoration of Anti-HER2CD4+Th1 Response in IBC Patients

In addition to the identification of a progressive loss of anti-HER2 CD4⁺ Th1 response across a tumorigenic continuum in HER2^{pos}-breast cancer, as taught by Datta, et al., the depressed anti-HER2 Th1 responses in HER2^{pos}-invasive breast cancer were differentially restored after HER2-pulsed type-1 polarized dendritic cell (“DC1”) vaccinations. The depressed responses were not restored following HER2-targeted therapy with trastuzumab and chemotherapy (“T/C”) or by other standard therapies such as surgical resection or radiation. The restored anti-HER2 Th1 responses appear to be durable for at least about six months or considerably longer.

Methods:

HER2⁺ IBC patients with residual disease following neoadjuvant therapy received adjuvant HER2-pulsed DC1 vaccines. Immune responses were generated from PBMCs pulsed with HER2 Class II peptides by measuring IFN-γ production via ELISPOT. Responses were evaluated on the three metrics of CD4⁺ Th1 response: (1) the overall anti-HER2 responsivity (responding to ≥1 peptide), (2) the number of reactive peptides (response repertoire), and (3) the cumulative response across the 6 HER2 peptides. Pre-vaccination Th1 responses were compared with 3-month and 6-month post-vaccination responses.

Datta, et al., describes the methods of making DC1 vaccines. See also, Koski, G. K., et al., J. Immonother. 35(1): 54 (2012) (“Koski, et al.”); Sharma, A., et al., Cancer 118(17):4354 (2012) (“Sharma, et al.”); Fracol, M., et al., Ann. Surg. Oncol. 20(10):3233 (2013); Lee, M. K. 4th, et al., Expert Rev. 8(11):e74698 (2013); Czerniecki, B. J., et al., Cancer Res. 67(4):1842 (2007); Czerniecki, B. J., et al., Cancer Res. 67(14):6531 (2007); and U.S. Published Application US 2013/0183343 A1. Briefly, patients' monocytes are first separated from other white blood cells by leukapheresis and elutriation. These monocytes are then cultured in serum-free medium (“SFM”) with granulocyte-macrophage colony-stimulating factor (“GM-CSF”) and interleukin (“IL”)-4 to become immature dendritic cells (“iDCs”). These cells are then preferably pulsed with six HER2 MHC class II binding peptides, and in the present case, binding peptides identified by SEQ ID NOS: 1-6, and then interferon (“IFN”)-γ and lipopolysaccharide (“LPS”) are added to complete the maturing and activation process to achieve full DC activation to DC1s before injecting back into the patient. See, Fracol, M., et al., Ann. Surg. Oncol. 20(10):3233 (2013). In the case of HLA-A2^{pos} patients, half of the cells are pulsed with a MHC class I binding peptide and the other half with a different MHC class 1 binding peptide.

Datta, et al. also describe blood tests/assays which generate a circulating anti-cancer CD4⁺ Th1 response (i.e., IFN-γ-secreting) and the resulting IFN-γ production is detected and measured. Such blood tests were performed on patients pre-DC1 vaccination, and 3-months and 6 months post-vaccination. In preferred embodiments, subject blood samples containing CD4⁺ Th1 cells and antigen-presenting cells or precursors thereof are pulsed with MHC class II immunogenic peptides based on the type of cancer the subject is afflicted with and which are capable of inducing an immune response in said subject. Preferably the antigen-presenting cells or precursors thereof are mature or immature dendritic cells or monocyte precursors thereof. In particularly preferred embodiments, the cancer is preferably HER2-expressing and the mammalian subject is preferably a human, and more preferably the cancer is HER2^{pos} breast cancer and the human subject is a female.

A preferred embodiment is provided for generating a circulating anti-HER2 CD4⁺ Th1 response in a mammalian subject by isolating unexpanded peripheral blood mononuclear cells (“PBMCs”) from a subject and pulsing the PBMCs with a composition comprising HER2-derived MHC class II antigenic binding peptides capable of generating an immune response in the subject. Without wishing to be bound by any particular theory, when the binding peptides are presented to CD4⁺ Th1 cells that are present in the PBMC sample they activate the CD4⁺ Th1 cells and the activated CD4⁺ Th1 cells produce interferon-γ (“IFN-γ”). DC1s (type-1 polarized dendritic cells) derived from precursor pluripotent monocytes contained in the subject's PBMC sample are antigen-presenting cells (“APCs”) which upon exposure to the binding peptides become antigen-loaded APCs which present the MHC class II antigen binding peptides to the subject's CD4⁺·Th1 cells in the sample thereby activating the CD4⁺ Th1 cells to produce/secrete IFN-γ. The IFN-γ thereby produced is subsequently measured for analysis.

In the present case, according to this preferred embodiment each patient's PBMC's were pulsed with 6 HER2-specific MHC class II peptides, in particular, those having sequences identified by SEQ ID NOs: 1-6. IFN-γ produced by anti-HER2 CD4⁺ Th1 cells was detected and measured via IFN-γ enzyme-linked immunospot (“ELISPOT”) assay.

In particularly preferred embodiments for HER2^{pos} cancers, DCs, immature or type-1 polarized DC1s, are pulsed with a composition comprising 6 MHC class II binding peptides derived from or based on HER2 that are capable of generating an immune response in a patient. HER2 MHC class II binding peptides or epitopes include:

Peptide 42-56:
(SEQ ID NO: 1)
HLDMLRHLYQGCQVV;
Peptide 98-114:
(SEQ ID NO: 2)
RLRIVRGTQLFEDNYAL;
Peptide 328-345:
(SEQ ID NO: 3)
TQRCEKCSKPCARVCYGL;
Peptide 776-790:
(SEQ ID NO: 4)
GVGSPYVSRLLGICL;
Peptide 927-941:
(SEQ ID NO: 5)
PAREIPDLLEKGERL;
and
Peptide 1166-1180:
(SEQ ID NO: 6)
TLERPKTLSPGKNGV.

In embodiments where donors have A2.1 blood type HER2 MHC class I peptides or epitopes include:

Peptide 369-377:
(SEQ ID NO: 7)
KIFGSLAFL;
and
Peptide 689-697:
(SEQ ID NO: 8)
RLLQETELV.

Datta, et al. also describe an alternate preferred embodiment, wherein a circulating anti HER2 CD4⁺ Th1 response is generated in a mammalian subject by co-culturing previously unstimulated purified CD4⁺ T-cells from a subject blood sample with autologous immature or mature dendritic cells (“iDCs” or mature “DCs”) pulsed with a composition comprising HER2-derived MHC class II antigenic binding peptides capable of generating an immune response in the subject. Without wishing to be bound by any particular theory, when the binding peptides are presented to CD4⁺ Th1 cells present in the T cell sample they activate the CD4⁺ Th1 cells and the activated CD4⁺ Th1 cells produce/secrete IFN-γ. The immature DCs are matured to DC1's, which present the MHC class II binding peptides to the subject's CD4⁺ Th1 cells that are present in the sample thereby activating the CD4⁺ Th1 cells to produce IFN-γ, which is subsequently measured for analysis.

In both alternate preferred embodiments for generating anti-HER2 immune response in a subject, IFN-γ produced by anti-HER2 CD4⁺ Th1 cells is detected and measured via IFN-γ enzyme-linked immunospot (“ELISPOT”) assay, although it should be understood by one skilled in the art that other detection methods may be used. For example, flow cytometry, enzyme-linked immunosorbant assay (“ELISA”), and immunofluorescence (“IF”) can be used for monitoring immune response. Alternatively, in instances of immune monitoring of patients, it can be advantageous to measure the ratio of IFN-γ to IL-10 as opposed to, or in addition to, a straight IFN-γ test such as ELISPOT which shows total CD4⁺ cell spots. Such testing would be particularly advantageous for patients at risk. Further, the use of immunofluorescence provides other ways to measure and visualize immune response via use of ELISPOT readers that read results by fluorescence. In such instances the results can be arranged to show 2, 3, or more cytokines/other secreted immune molecules, each showed in a different color, in the same patient sample. In the present case IFN-γ ELISPOT was used.

Although a presently preferred embodiment features six HER2 MHC class II binding peptides/epitopes, other possible MHC class II HER2 peptides can be used in the present embodiments in that any components of the entire HER2 molecule can be used as a source for other binding peptides so long as they are sufficiently immunologically active in patients.

### Results:

Responsivity: Pre-vaccination, only one IBC patient produced an immune response, defined as >20 SFC/10⁶ cells in an experimental well after subtracting unstimulated background. Compared with pre-vaccination results, all vaccinated IBC patients produced an immune response, defined as >2-fold increase in anti-HER2 IFN-γ^{pos}Th1 responses.

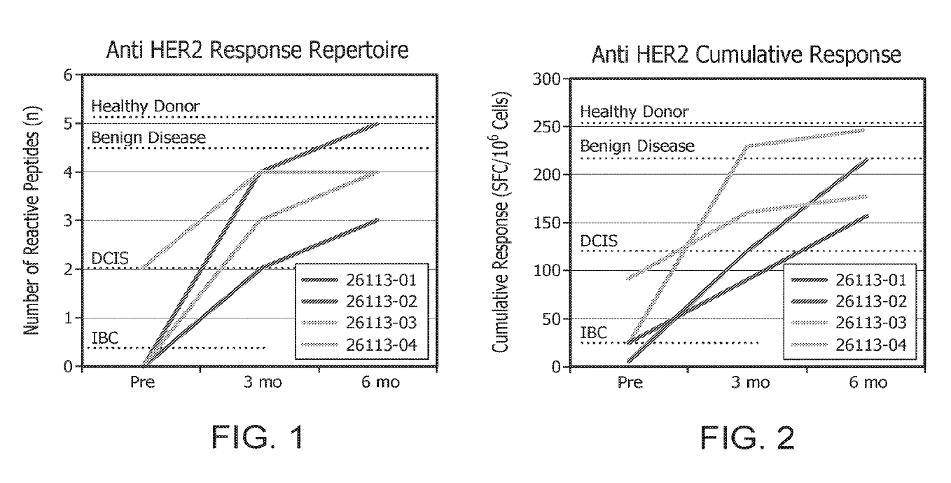

Response Repertoire: FIG. 1 shows mean repertoire increased from 0.5±1 peptides pre-vaccination to 3.25±0.96 peptides at 3 months (p=0.01) and 4±0.8 peptides at 6 months (p=0.01) in the IBC patients.

Cumulative response: FIG. 2 shows mean cumulative response in the patients improved from 36.5±38.3 SFC/10⁶ pre-vaccination to 151.0±60.0 SFC/10⁶ at 3 months (p=0.04) and 198.4±39.7 SFC/10⁶ at 6 months (p=0.02).

There are many other HER2^{pos} solid cancers in addition to breast cancer, such as, for example, brain, bladder, esophagus, lung, pancreas, liver, prostate, ovarian, colorectal, and gastric, and others, for which the materials and methods of the embodiments described herein can be used for diagnosis and treatment. Therefore the six anti-HER2 binding peptides described above may be used in accordance with the herein embodiments to generate immune responses capable of detection and useful for diagnostics for these and other HER2-expressing cancers.

Vaccines can be developed to target HER2-expressing tumors using the same anti-HER2 binding peptides described above or may employ any composition of HER2 that is immunogenic such as, for example, DNA, RNA, peptides, or proteins or components thereof such as the ICD and ECD domains. For example, subjects can be vaccinated against the whole HER2 protein and the six above-referenced binding peptides can be used to monitor the patient's immune response. Similarly vaccines can be developed for other types of cancer such as other members of the HER2 family which includes HER1, HER3, and c-MET.

Although the present preferred embodiments are directed to treating and diagnosing HER2^{pos} breast cancer in women it should be readily appreciated by the skilled artisan that the present embodiments are not limited to female humans. The presently preferred embodiments includes male humans, for example, HER2-expressing prostate cancer, as well as other mammalian subjects

The identified anti-HER2 CD4⁺ Th1 response decrement allows the detected immune response generated in such blood tests to be used as a cancer diagnostic/response predictor alone or, as in the example here, in tandem with the use of specialized vaccines to restore a patient's immune response. The preferred embodiments described herein thus shift the focus of cancer diagnosis and therapy to patient immunity and use of blood tests to determine and/or predict the immune response against a cancer, including patients at risk for recurrence, as opposed to diagnosis and treatment methods that rely on identification of tumor cells.

### In Vitro Expansion of HER2-Specific Th1 Cells

### Methods

In vitro, HER2-specific Th1 cells were generated by co-culture with HER2-peptide pulsed DC1s and expanded using IL-2 alone or IL-2, IL-7, and IL-15. Th1 cells were subsequently expanded either by repeat HER2-peptide pulsed DC1 co-culture or via anti-CD3/CD28 stimulation. Fold expansion was defined as: (#T-cells post expansion/#T-cells pre expansion); specificity was measured by antigen specific IFN-γ production by ELISA.

The present embodiments related to T cell expansion are in no way limited to CD4⁺ T cells. Thus the present embodiments provide methods for growing chimeric antigen receptor T cells (“CART cells”), cytotoxic T lymphocytes (CD8⁺'s), as well as all other kinds of T cells. See, for example, Datta, J., et al., Cancer Immunol. Res. 3:455-463 (2015).

Present embodiments relate to replicating the environment of the lymph node for generating a therapeutic amount of antigen-specific T cells, either helper (CD4⁺) or cytotoxic (CD8⁺), for adoptive therapy for cancer or other conditions. The expanded antigen-specific T lymphocytes can also be used for the identification of epitopes on target antigens to foster the production of peptide-based vaccines.

A present embodiment provides an in vitro environment that replicates the environment of the lymph node. In that embodiment, replication of the lymph node comprises supplying one or more of the following elements to the culture conditions: type 1 dendritic cells, IL-15, IL-7, and IL-2.

Type 1 dendritic cells process and present peptide antigens to T cells and supply so-called “costimulatory molecules” including surface-expressed CD80 and CD86 (which bind to CD28 counter-receptor on T cells), as well as CD40 (which interacts with CD40L on T cells). In addition, the DCs produce soluble factors such as Interleukin-12 (“IL-12”) which supports long life (anti-apoptotic factor) as well as IFN-γ production (T cell function). DCs produce a number of other factors critical to T cell development. DCs are normally found in lymph nodes and are of known importance to T cell activation and expansion.

IL-15 is a T cell growth activation and survival factor. IL-15 is produced by fibroblasts, dendritic cells and macrophages.

IL-7 is a factor produced by stromal epithelial cells in lymph nodes. IL-7 is essential for lymphocyte proliferation and survival.

IL2 is the principal T cell growth and proliferation factor.

Accordingly, the embodiments provide compositions and methods for combining the particular cytokines and type of dendritic cells while also using particular timing and sequence of lymphocyte addition to generate desirable T cells. In preferred embodiments, T cells are expanded to a level necessary for adoptive therapy and epitope mapping studies while maintaining antigen specificity and cellular function.

### Sources of T Cells

Prior to expansion, a source of T cells is obtained from a subject. Examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, and tumors. In certain embodiments herein, any number of T cell lines available in the art, may be used. In certain embodiments, T cells can be obtained from a unit of blood collected from a subject using any number of techniques known to the skilled artisan, such as ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment, the cells are washed with phosphate buffered saline (“PBS”). In an alternative embodiment, the wash solution lacks calcium and may lack magnesium or may lack many if not all divalent cations. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment. T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In another embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells for stimulation can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

Activation and Expansion of T Cells

Generally, T cells of the embodiments are expanded under conditions that replicate the lymph node. In one embodiment, replication of the lymph node comprises supplying one or more of the following elements to the culture conditions: type 1 dendritic cells, IL-15, IL-7, and IL-2. In one embodiment, antigen-specific T cells can be expanded in the presence of one or more of type 1 dendritic cells, IL-15, IL-7, and IL-2.

In one embodiment, the T cells may be stimulated as described herein, such as by contacting with a DC. The DC is able to provide supply a costimulatory molecule to the T cell. After the T cells are contacted with DCs, the T cells are cultured in the presence of IL-15, IL-7, and IL-2.

In some instances, T cells are co-cultured with a mixture comprising one or more of DCs, IL-15, IL-7, and IL-2. In one of the present embodiments, the mixture may be co-cultured for several hours (about 3 hours) to about 14 days or any hourly integer value in between. In another embodiment, the mixture may be cultured for 21 days. In one embodiment T cells are cultured for about eight days. In another embodiment, T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15) that may contain factors necessary for proliferation and viability, including serum (e.g., fetal bovine or human serum), IL-2, insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol.

Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.).

In one embodiment, the T cells are expanded to a level necessary for adoptive therapy and epitope mapping studies while maintaining antigen specificity and cellular function. Accordingly, any cell number is within the context of the present embodiments. Cells stimulated by the present methods are activated as shown by the induction of signal transduction, expression of cell surface markers and/or proliferation. One such marker appropriate for CD4⁺ T cells is IFN-γ production which is an important immunomodulating molecule. The production of IFN-γ is extremely beneficial in amplifying the immune response.

With respect to T cells, the T cell populations resulting from the various expansion methodologies described herein may have a variety of specific phenotypic properties, depending on the conditions employed. Such phenotypic properties include enhanced expression of CD25, CD154, IFN-γ and GM-CSF, as well as altered expression of CD137, CD134, CD62L, and CD49d. The ability to differentially control the expression of these moieties may be very important. For example, higher levels of surface expression of CD154 on “tailored T cells,” through contact with CD40 molecules expressed on antigen-presenting cells (such as dendritic cells, monocytes, and even leukemic B cells or lymphomas), will enhance antigen presentation and immune function. Such strategies are currently being employed by various companies to ligate CD40 via antibodies or recombinant CD40L. The approach described herein permits this same signal to be delivered in a more physiological manner, e.g., by the T cell. The ability to increase IFN-γ secretion by tailoring the T cell activation process could help promote the generation of Th1-type immune responses, important for anti-tumor and anti-viral responses. Like CD154, increased expression of GM-CSF can serve to enhance APC function, particularly through its effect on promoting the maturation of APC progenitors into more functionally competent APC, such as dendritic cells. Altering the expression of CD137 and CD134 can affect a T cell's ability to resist or be susceptible to apoptotic signals. Controlling the expression of adhesion/homing receptors, such as CD62L and/or CD49d and/or CCR7 may determine the ability of infused T cells to home to lymphoid organs, sites of infection, or tumor sites.

The phenotypic properties of T cell populations can be monitored by a variety of methods including standard flow cytometry methods and ELISA methods known by those skilled in the art.

Those of ordinary skill in the art will readily appreciate that the cell stimulation methodologies described herein may be carried out in a variety of environments (i.e., containers). For example, such containers may be culture flasks, culture bags, or any container capable of holding cells, preferably in a sterile environment. In one embodiment a bioreactor is also useful. For example, several manufacturers currently make devices that can be used to grow cells and be used in combination with the methods of the present embodiments. See for example, Celdyne Corp., Houston, Tex.; Unisyn Technologies, Hopkinton, Mass.; Synthecon, Inc., Houston, Tex.; Aastrom Biosciences, Inc., Ann Arbor, Mich.; Wave Biotech LLC, Bedminster, N.J. Further, patents covering such bioreactors include U.S. Pat. Nos. 6,096,532; 5,985,653; 5,888,807; and 5,190,878, which are incorporated herein by reference.

In one embodiment, a bioreactor with a base rocker platform is used, for example “The Wave” (Wave Biotech LLC, Bedminster, N.J.), that allows for varying rates of rocking and at a variety of different rocking angles. The skilled artisan will recognize that any platform that allows for the appropriate motion for optimal expansion of the cells is within the context of the present embodiments. In certain embodiments, the methods of stimulation and expansion of the present embodiments provide for rocking the culture container during the process of culturing at 1, 2, 3, 4, 5, 6, 7, 8, 9, 10, 11, 12, 13, 14, 15, 16, 17, 18, 19, or 20 rocks per minute. In certain embodiments, the methods of stimulation and expansion of the present embodiments provide for the angle of the rocking platform to be set at 1.5°, 2°, 2.5°, 3°, 3.5°, 4°, 4.5°, 5°, 5.5°, 6°, 6.5°, 7°, 7.5°, 8°, 8.5°, or 9.0°.

In certain embodiments, the capacity of the bioreactor container ranges from about 0.1 liter to about 200 liters of medium. The skilled artisan will readily appreciate that the volume used for culture will vary depending on the number of starting cells and on the final number of cells desired. In particular embodiments, the cells of the present embodiments, such as T cells are seeded at an initial concentration of about 0.2×10⁶ cells/ml to about 5×10⁶ cells/ml, and any concentration therebetween. In one particular embodiment, the cells may be cultured initially in a static environment and transferred to a bioreactor on a rocking platform after 1, 2, 3, 4, 5, 6, 7, 8, or more days of culture. In a related embodiment, the entire process of stimulation, activation, and expansion takes place in a bioreactor comprising a rocking platform and an integrated magnet, as described above. Illustrative bioreactors include, but are not limited to, “The Wave”.

In one particular embodiment, the cell stimulation methods are carried out in a closed system, such as a bioreactor, that allows for perfusion of medium at varying rates, such as from about 0.1 ml/minute to about 10 ml/minute. Accordingly, in certain embodiments, the container of such a closed system comprises an outlet filter, an inlet filter, and a sampling port for sterile transfer to and from the closed system. In other embodiments, the container of such a closed system comprises a syringe pump and control for sterile transfer to and from the closed system. Further embodiments provide for a mechanism, such as a load cell, for controlling media in-put and out-put by continuous monitoring of the weight of the bioreactor container. In one embodiment the system comprises a gas manifold. In another embodiment, the bioreactor of the present embodiments comprises a CO₂ gas mix rack that supplies a mixture of ambient air and CO₂ to the bioreactor container and maintains the container at positive pressure. In another embodiment, the bioreactor of the present embodiments comprises a variable heating element.

In an embodiment, media is allowed to enter the container starting on day 2, 3, 4, 5, or 6 at about 0.5 to 5.0 liters per day until the desired final volume is achieved. In another embodiment, media enters the container at 2 liters per day starting at day 4, until the volume reaches 10 liters. Once desired volume is achieved, perfusion of media can be initiated. In certain embodiments, perfusion of media through the system is initiated on about day 2, 3, 4, 5, 6, 7, 8, 9, 10, 11, or 12 of culture. In another embodiment, perfusion is initiated when the volume is at about 0.1 liter to about 200 liters of media. In one particular embodiment, perfusion is initiated when the final volume is at 4, 5, 6, 7, 8, 9, 10, or 20 liters or higher volume. The rate of perfusion can be from about 0.5 ml/minute to about 10 ml/minute. In certain embodiments, the perfusion rate is about 1, 2, 2.5, 3, 3.5, 4, 4.5, 5, 5.5, 6, 6.5, 7, 7.5, or 8.0 mls/minute.

In a further embodiment, the cells, such as T cells, are cultured for up to 5 days in a closed, static system and then transferred to a closed system that comprises a rocking element to allow rocking of the culture container at varying speeds.

In certain aspects, the methodologies of the present embodiments provide for the expansion of cells, such as T cells, to a concentration of about between 6×10⁶ cell/ml and about 90×10⁶ cells/ml in less than about two weeks. In particular the methodologies herein provide for the expansion of T cells to a concentration of about 10, 15, 20, 25, 30, 35, 40, 45, 50, 55, 60, 65, 70, 75, 80, or 85×10⁶ cells/ml and all concentrations therein. In certain embodiments, the cells reach a desired concentration, such as any of those listed above, by about day 5, 6, 7, 8, 9, 10, 11, or 12 of culture. In one embodiment, the T cells expand by at least about 1.5 fold in about 24 hours from about day 4 to about day 12 of culture. In one embodiment, the cells, such as T cells, expand from a starting number of cells of about 100×10⁶ to a total of about 500×10⁹ cells in less than about two weeks. In further embodiments, the T cells expand from a starting number of cells of about 500×10⁶ to a total of about 500×10⁹ cells in less than about two weeks. In related embodiments, the cells expand from a starting number of about 100-500×10⁶ to a total of about 200, 300, or 400×10⁹ cells in less than about two weeks.

Therapy

In certain embodiments, a population of T cells is first contacted with antigen, for example, a tumor target antigen, and then subjected to a mixture of the embodiments comprising one or more of DCs, IL-15, IL-7, and IL-2. In one particular embodiment, the antigen-specific T cells are induced by vaccination of a patient with a particular antigen, either alone or in conjunction with an adjuvant or pulsed on dendritic cells. Antigen-specific cells for use in expansion using the stimulation method of the embodiments may also be generated in vitro.

Another aspect of the present embodiments provides a method for expanding antigen specific T cells, comprising contacting a population of T cells with an antigen for a time sufficient to induce activation of T cells specific to said antigen; contacting said population of antigen-specific T cells ex vivo with a mixture comprising one or more of DCs, IL-15, IL-7, and IL-2 under conditions and for time sufficient to induce proliferation of T cells specific to said antigen, thereby expanding antigen-specific T cells. In one embodiment, the antigen is a tumor target antigen. In another embodiment, the antigen is pulsed on or expressed by an antigen-presenting cell. In another embodiment, the population of T cells is contacted with said antigen ex vivo. In another embodiment, the method comprises at least one round of peptide-MHC tetramer sorting of said antigen-specific T cells. In certain embodiments, the method further comprises at least one round of peptide-MHC tetramer magnetic selection of said antigen-specific T cells.

Another aspect of the embodiments herein provides a method for the treatment of cancer comprising administering to a cancer patient antigen-specific T cells expanded according to the methods provided herein.

The T cells generated according to the present methods can also be used to treat autoimmune diseases. Examples of autoimmune disease include but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

The cells generated according to the present methods can also be used to treat inflammatory disorders. Examples of inflammatory disorders include but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

The present embodiments also provide methods for preventing, inhibiting, or reducing the presence of a cancer or malignant cells in an animal, which comprise administering to an animal an anti-cancer effective amount of the anti-tumor cells of the present embodiments.

The cancers contemplated by the present embodiments, against which the immune response is induced, or which is to be prevented, inhibited, or reduced in presence, may include but are not limited to melanoma, non-Hodgkin's lymphoma, Hodgkin's disease, leukemia, plasmocytoma, sarcoma, glioma, thymoma, breast cancer, prostate cancer, colorectal cancer, kidney cancer, renal cell carcinoma, pancreatic cancer, esophageal cancer, brain cancer, lung cancer, liver cancer, ovarian cancer, cervical cancer, multiple myeloma, hepatocellular carcinoma, nasopharyngeal carcinoma, ALL, AML, CML, CLL, and other neoplasms known in the art.

Alternatively, compositions as described herein can be used to induce or enhance responsiveness to pathogenic organisms, such as viruses, (e.g., single stranded RNA viruses, single stranded DNA viruses, double-stranded DNA viruses, HIV, hepatitis A, B, and C virus, HSV, CMV, EBV, HPV), parasites (e.g., protozoan and metazoan pathogens such as Plasmodia species, Leishmania species, Schistosoma species, Trypanosoma species), bacteria (e.g., Mycobacteria, Salmonella, Streptococci, E. coli, Staphylococci), fungi (e.g., Candida species, Aspergillus species) and Pneumocystis carinii.

The immune response induced in an animal by administering the subject compositions may include cellular immune responses mediated by CD8⁺ T cells, capable of killing tumor and infected cells, and CD4⁺ T cell responses. Humoral immune responses, mediated primarily by B cells that produce antibodies following activation by CD4⁺ T cells, may also be induced. A variety of techniques may be used for analyzing the type of immune responses induced by the compositions of the present embodiments, which are well described in the art.

When “an immunologically effective amount,” “an anti-tumor effective amount,” “a tumor-inhibiting effective amount,” or “therapeutic amount” is indicated, the precise amount of the compositions of the present embodiments to be administered can be determined by a physician with consideration of individual differences in age, weight, tumor size, extent of infection or metastasis, and condition of the patient. It can generally be stated that a pharmaceutical composition comprising the subject cells of the present embodiments, may be administered at a dosage to be determined during appropriate clinical trials. Cells of the present embodiments may also be administered multiple times at these dosages. The optimal dosage and treatment regime for a particular patient can readily be determined by one skilled in the art of medicine by monitoring the patient for signs of disease and adjusting the treatment accordingly.

Cells of the present embodiments can be administered in dosages and routes and at times to be determined in appropriate clinical trials. Cell compositions may be administered multiple times at dosages within these ranges. The cells of the present embodiments may be combined with other methods. The cells of the present embodiments for administration may be autologous, allogeniec or xenogenic to the patient undergoing therapy. If desired, the treatment may also include administration of mitogens (e.g., PHA) or lymphokines, cytokines, and/or chemokines (e.g., GM-CSF, IL-4, IL-13, Flt3-L, RANTES, MIP1-α, etc.) as described herein to enhance induction of the immune response.

The administration of the cells of the present embodiments may be carried out in any convenient manner. The cells of the present embodiments may also be administered to a patient subcutaneously, intradermally, intramuscularly, by intravenous (“i.v.”) injection, or intraperitoneally. In some instances, the cells are administered to a patient by intradermal or subcutaneous injection. In other instances, the cells of the embodiments are administered by i.v. injection. In other instances, the cells of the embodiments are injected directly into a tumor or lymph node.

The cells of the present embodiments can also be administered using any number of matrices. The present embodiments utilize such matrices within the novel context of acting as an artificial lymphoid organ to support, maintain, or modulate the immune system, typically through modulation of T cells. Accordingly, the present embodiments can utilize those matrix compositions and formulations which have demonstrated utility in tissue engineering. Accordingly, the type of matrix that may be used in the compositions, devices and methods of the present embodiments is virtually limitless and may include both biological and synthetic matrices. In one particular example, the compositions and devices set forth by U.S. Pat. No. 5,980,889; 5,913,998; 5,902,745; 5,843,069; 5,787,900; or 5,626,561 are utilized; these patents are incorporated herein by reference in their entirety. Matrices comprise features commonly associated with being biocompatible when administered to a mammalian host. Matrices may be formed from natural and/or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release of seeded cells or produced cytokine or other active agent. In certain embodiments, the matrix is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current embodiments are not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

In one aspect of the present embodiments, the expanded cells herein can be used in vivo as an adjuvant as described in U.S. Pat. No. 6,464,973. In a further embodiment, the cells can be used as a vaccine to induce an immune response in vivo against an antigen of interest such as those described herein (e.g., tumor antigens, viral antigens, autoantigens, etc). In one embodiment the cells can be used to generate an immune response in vivo, either administered alone or in combination with other immune regulators and in combination with other known therapies.

Identification of Epitopes

In one embodiment there is provided compositions and methods to expand antigen-specific T cells. The antigen-specific T cells can be expanded by steps which comprise contacting the T cell with one or more of DCs, IL-15, IL-7, and IL-2. The expanded T cells can be used to identify antigen-specific T cell receptors (“TCRs”) and epitopes derived therefrom. For example, TCRs from the expanded T cells can be cloned. The cloned TCRs present a promising tool for the development of specific adoptive T cell therapies to treat a desired disease or disorder. For example, the cloned TCRs can be used to generate peptides/antigens useful for vaccines.

In addition to their role in combating infections, T cells have also been implicated in the destruction of cancerous cells. Autoimmune disorders have also been linked to antigen-specific T cell attack against various parts of the body. One of the major problems hampering the understanding of and intervention on the mechanisms involved in these disorders is the difficulty in identifying T cells specific for the antigen to be studied.

TCRs are closely related to antibody molecules in structure, and they are involved in antigen binding although, unlike antibodies, they do not recognize free antigen; instead, they bind antigen fragments which are bound and presented by antigen-presenting molecules. An important group of antigen-presenting molecules are the MHC class I and class II molecules that present antigenic peptides and protein fragments to T cells.

Variability in the antigen binding site of a TCR is created in a fashion similar to the antigen binding site of antibodies, and also provides specificity for a vast number of different antigens. Diversity occurs in the complementarity determining regions (“CDRs”) in the N-terminal domains of the disulfide-linked alpha (α) and beta (β), or gamma (γ) and delta (Δ), polypeptides of the TCR. The CDR loops are clustered together to form an MHC-antigen-binding site analogous to the antigen-binding site of antibodies, although in TCRs, the various chains each contain two additional hypervariable loops as compared to antibodies. TCR diversity for specific antigens is also directly related to the MHC molecule on the APC's surface to which the antigen is bound and presented to the TCR.

In the embodiments described elsewhere herein, a peptide can be located within the MHC molecule of a dendritic cell in order to generate suitable T-cells. In some embodiments, the MHC molecule is loaded with the peptide extracellularly by incubating cells at 37° C., 5% CO₂ for 4 hours with varying concentrations of peptide, then washed once in serum-free RPMI. However, in alternative embodiments, the antigen presenting cells are transfected with a polynucleotide encoding a fusion protein comprising the peptide connected to at least an MHC Class I molecule alpha chain by a flexible linker peptide. Thus, when expressed, the fusion protein results in the peptide occupying the MHC Class I binding groove. Suitable MHC Class I molecules and costimulatory molecules are available from public databases. Further details of the synthesis of such a fusion molecule may be found in Mottez, E., et al, J Exp Med., 181(2):493-502 (Feb. 1, 1995). The advantage of expressing a fusion protein of the peptide and the MHC molecule is that a much higher concentration of peptide is displayed on the surface of the antigen presenting cells.

In some situations in the preparation of T cells, it is preferred that there is an HLA match between the antigen presenting cells and the T cells. That is to say the antigen-presenting cells display an MHC molecule of an allele for which the donor of the T cells is HLA positive. In some embodiments, this is achieved by obtaining the antigen presenting cells from a first individual and the T cells from a second individual wherein the first and second individuals have an HLA match.

However, in alternative embodiments, the antigen presenting cells and the T cells are obtained from the same individual but the antigen presenting cells are transfected with polynucleotides encoding the MHC molecule of a similar HLA allele. In some embodiments, the polynucleotide encodes a protein which encodes the MHC molecule connected to the peptide via a linker. There are numerous HLA Class I alleles in humans and the MHC molecule displayed by the antigen presenting cells, may, in principle, be of any of these alleles. However, since the HLA-A*0201 allele is particularly prevalent, it is preferred that the MHC molecule be of this allele. However, any HLA-A2 allele is usable or other alleles such as HLA-A1, HLA-A3, HLA-A 11 and HLA-A24 may be used instead.

In further embodiments there is provided a method for preparing T cells suitable for delivery to a patient suffering from cancer. The method comprises providing dendritic cells expressing an HLA molecule of a first HLA allele and locating a peptide in the binding groove of the HLA molecule. The peptide may or may not be a peptide of the present embodiments. T cells are then primed with the dendritic cells, the T cells being obtained or obtainable from an individual who is HLA matched for a first HLA allele. As described elsewhere herein, the dendritic cells may either be obtained from a first donor individual and the T cells from a second donor individual wherein the first and second donor individuals are HLA matched. The advantage of using dendritic cells, rather than non-professional antigen presenting cells is that it results in a much higher stimulus of the T cells.

In these embodiments, it is preferred that the peptide is a cell type specific peptide, that is to say a peptide that is obtained from a protein which is only expressed, or is expressed at a much higher level (e.g. at least 10× higher concentration) in specific cells than in other cell types.

The T cells prepared in accordance with the embodiments herein described are administered to patients in order to treat cancer in the patients. In principle, the T cells of the embodiments are capable of being used for the treatment of many different types of cancer including leukemia, lymphomas such as non-Hodgkin lymphoma, multiple myeloma and the like.

Thus, in some embodiments, pharmaceutical preparations are provided comprising a T cell of the present embodiments and a pharmaceutically acceptable carrier, diluent or excipient, further details of which may be found in Remington's Pharmaceutical Sciences in US Pharmacopeia, 1984 Mack Publishing Company, Easton, Pa., USA.

As discussed elsewhere herein, the HLA allele of the MHC molecule used to present the peptide to the T cells is an HLA allele also expressed by the patient and therefore when the T cells are administered to the patient, they recognize the peptide displayed on MHC molecules of that HLA allele.

In some alternative embodiments, multiple sets (e.g. 2 or 3 sets) of T-cells are provided, each T cell being specific for a different peptide. In each case, the T cells are allogeneic, as described elsewhere herein, that is to say the HLA allele of the MHC molecule on which the peptide is displayed during preparation of the T cells is an HLA allele which is not expressed in the donor individual from whom the T cells are obtained. The peptides may all be from the same cell specific protein or may be from different proteins but specific for the same cell type. In some embodiments, the multiple sets of T cells are administered simultaneously but in other embodiments they are administered sequentially.

Reference has been made to the expansion antigen-specific T cells. However, it is to be appreciated that the important feature of T cells is the T cell receptor (“TCR”) which is displayed on the T cells and, more specifically, the specificity of the T cell receptor for the complex of the peptide and the MHC molecule. Therefore, in some alternative embodiments, following the preparation of T cells as described elsewhere herein, the T cell receptors of T-cells specific for a certain peptide when complexed with an MHC molecule of a particular allele are harvested and sequenced. A cDNA sequence encoding the T cell receptor is then generated and which can be used to express the T cell receptor recombinantly in a T-cell (e.g. the patient's own T cells or T cells from a donor). For example, the cDNA may be incorporated into a vector such as a viral vector (e.g. a retroviral vector), lentiviral vector, adenoviral vector or a vaccinia vector. Alternatively, a non-viral approach may be followed such as using naked DNA or lipoplexes and polyplexes or mRNA in order to transfect a T cell.

Thus a T cell which is “obtainable” from a donor individual includes a T cell which is obtained recombinantly in the manner described elsewhere herein because the recombinantly expressed TCR is naturally produced.

Since transfected T cells also display their endogenous TCRs, it is preferred that the T cells are pre-selected, prior to transfection, to eliminate T cells that would give rise to graft-versus-host disease. In some embodiments, T cells are pre-selected such that the specificity of their endogenous TCRs is known. For instance, T cells are selected which are reactive with glypican-3. In other embodiments, T cells are obtained from the patient and thus are naturally tolerized for the patient. This approach can only be adopted where the T cells of the patient are healthy.

In some alternative embodiments, the T cell receptor, as a whole, is not recombinantly expressed but rather the regions of the T cell receptor which are responsible for its binding specificity are incorporated into a structure which maintains the confirmation of these regions. More specifically, complementarity determining regions (CDRs) 1 to 3 of the T cell receptor are sequenced and these sequences are maintained in the same conformation in the recombinant protein.

In one embodiment, the expanded T cells provide a source for cloning TCRs and epitopes/antigens associated therewith. The epitopes/antigens identified can be used to generate a vaccine. In one embodiment the vaccination antigens can be constructed by modifying a polypeptide (e.g. the target antigen) at specific amino acid positions identified by epitope mapping. Thus the present embodiments include method of identifying relevant positions for modification in the target antigen by epitope mapping, modifying the target antigen at relevant positions to produce variants, and including the variants in separate candidate preparations.

Vaccination antigen polypeptides may be epitope mapped by a number of methods, including those disclosed in detail in WO00/26230 and WO01/83559. In brief, these methods use a database of epitope patterns (determined from an input of peptide sequences, known to bind specifically to anti-protein antibodies) and an algorithm to analyze 3-D structure of a given protein against the epitope pattern database. This will determine the possible epitopes on that protein, and the preference of each amino acid in the protein sequence to be part of epitopes.

In another aspect, methods are provided for identifying candidate MHC class II epitopes. In certain embodiments, candidate epitopes can be identified using a computer-implemented algorithm for candidate epitope identification. Such computer programs include, for example, the TEPITOPE program (see, e.g., Hammer et al., Adv. Immunol. 0.66:67-100 (1997); Sturniolo et al., Nat. Biotechnol. 17:555-61 (1999); Manici et al., J. Exp. Med. 189:871-76 (1999); de Lalla et al., J. Immunol. 163:1725-29 (1999); Cochlovius et al., J. Immunol. 165:4731-41 (2000)), as well as other computer implemented algorithms.

The computer-implemented algorithm for candidate epitope identification can identify candidate epitopes in, for example, a single protein, in a very large protein, in a group of related proteins (e.g., homologs, orthologs, or polymorphic variants), in a mixtures of unrelated proteins, in proteins of a tissue or organ, or in a proteome of an organism. Using this approach, it can be possible to interrogate complex tissues or organisms based on sequence information for expressed proteins (e.g., from deduced open reading frame or a cDNA library), in addition to analysis of known candidate molecular targets, as an efficient, sensitive and specific approach to identification of T cell epitopes.

Following identification of candidate epitopes, peptides or pools of peptides can be formed that correspond to the candidate epitope(s). For example, once a candidate epitope is identified, overlapping peptides can be prepared that span the candidate epitope, or portions thereof, to confirm binding of the epitope by the MHC class II molecule, and, as necessary, to refine the identification of that epitope. Alternatively, pools of peptides can be prepared including a plurality of candidate epitopes identified using a computer-implemented algorithm for candidate epitope identification.

T Cell Epitope Peptides

T cell epitope peptides/binding peptides/peptides are short peptides that can be derived from a protein antigen. Antigen presenting cells can directly bind antigen via surface MHC molecules and/or internalize antigen and process it into short fragments which are capable of binding MHC molecules. The specificity of peptide binding to the MHC depends on specific interactions between the peptide and the peptide-binding groove of the particular MHC molecule.

Peptides which bind to MHC class I molecules are usually between 6 and 30, more usually between 7 and 20 amino or between 8 and 15 amino acids in length. The amino-terminal amine group of the peptide makes contact with an invariant site at one end of the peptide groove, and the carboxylate group at the carboxy terminus binds to an invariant site at the other end of the groove. Thus, typically, such peptides have a hydrophobic or basic carboxy terminus and an absence of proline in the extreme amino terminus. The peptide is in an extended confirmation along the groove with further contacts between main-chain atoms and conserved amino acid side chains that line the groove. Variations in peptide length are accommodated by a kinking in the peptide backbone, often at proline or glycine residues.

Peptides which bind to MHC class II molecules are usually at least 10 amino acids, for example about 13-18 amino acids in length, and can be much longer. These peptides lie in an extended confirmation along the MHC II peptide-binding groove which is open at both ends. The peptide is held in place mainly by main-chain atom contacts with conserved residues that line the peptide-binding groove.

The peptides used in the embodiments herein may be made using chemical methods. For example, peptides can be synthesized by solid phase techniques (Roberge, J. Y. et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptides may alternatively be made by recombinant means or by cleavage from a longer polypeptide. For example, the peptides may be obtained by cleavage from full-length glypican-3 protein. The composition of a peptide may be confirmed by amino acid analysis or sequencing.

The peptides used in the herein embodiments can be tested in an antigen presentation system which comprises antigen presenting cells and T cells. For example, the antigen presentation system may be a murine splenocyte preparation, a preparation of human cells from tonsil or PBMCs. Alternatively, the antigen presentation system may comprise a particular T cell line/clone and/or a particular antigen presenting cell type.

T cell activation may be measured via T cell proliferation (for example using ³H-thymidine incorporation) or cytokine production. Activation of TH1-type CD4⁺ T cells can, for example be detected via IFN-γ production which may be detected by standard techniques, such as an ELISPOT assay.

Measurement of antigen-specific T cells during an immune response are important parameters in vaccine development, autologous cancer therapy, transplantation, infectious diseases, inflammation, autoimmunity, toxicity studies, and the like. Peptide libraries are crucial reagents in monitoring of antigen-specific T cells. The present embodiments provide improved methods for the use of a peptide library in analysis of T cells in samples including diagnostic, prognostic and immune monitoring methods. Furthermore the use of a peptide library in anti-tumor therapy are described elsewhere herein, including isolation of antigen-specific T cells capable of inactivation or elimination of undesirable target cells or isolation of specific T cells capable of regulation of other immune cells. The present embodiments also relate to MHC multimers comprising one or more tumor derived peptides.

The identification of particular antigenic peptides provides new opportunities for the development of diagnostic and therapeutic strategies against cancer. Advantageously, identification of novel T cell epitopes enable the production of MHC class I and class II multimers, tetramers and pentamers, useful as analytical tools delivering both increased sensitivity of immuno-monitoring. In addition, the detection of antigen specific CTL in the periphery of individuals at risk of disease recurrence is a useful diagnostic tool.

The embodiments also provide compositions and methods for identifying peptides useful for cancer therapy. Peptide sequences from a candidate protein predicted to bind to HLA-A*0201 can be identified by a computer algorithm. Peptides are selected for synthesis according to predicted affinity with a cut-off value of 500 nM or less, but also higher values may be chosen. Peptides are synthesized and binding to HLA-A*0201 can be confirmed using biochemical assays. Peptide binding is compared with the binding achieved with a pass/fail control peptide, designated 100%, and with a positive control peptide. Corresponding HLA-A*0201-peptide multimers are also synthesized for peptides with a binding affinity above the pass/fail control peptide. These peptides are tested for the ability to generate a T cell line specifically reacting with the specific peptide-HLA-A*0201 complex. The cell line can be referred to as multimer-, tetramer-, or pentamer-positive T cells. Multimer positive cells indicate a high immunogenicity for the corresponding peptide. Additional responses can be measured to assess production of the cytokine INF-γ, degranulation and killing of target cells.

In some embodiments, the peptides can be administered directly to a patient as a vaccine. Thus the peptides are immunogenic epitopes of specific proteins and are used in order to elicit a T cell response to their respective proteins. In some embodiments, the polypeptide of the embodiments is administered directly to a patient as a vaccine. For example, in a patient that has leukemia, a polypeptide comprising a peptide from a hematopoietic cell specific protein is administered to the patient in order to elicit a T cell response to the protein. The T cell response leads to death of hematopoietic cells, including the cancerous cells, but is specific to these cells and does not result in an immune response to other cell types.

It is also to be noted that, in many patients, directly administering such a peptide will not elicit a T cell response because the cell specific protein is a “self-protein” and any T cells that are capable of binding the polypeptide when presented on an MHC molecule of the HLA alleles of the patient are tolerized. That is to say T cells that would be reactive are either destroyed in the thymus of the patient during the selection process or are inactivated through central or peripheral tolerance mechanisms. Therefore, it is preferred that the peptides herein are used to generate T cells obtained, or obtainable, from an allogeneic donor individual. This individual should preferably be HLA negative for an HLA allele of which the patient is HLA positive. For example, if the patient is HLA positive for the HLA allele HLA-A*0201 then T cells are obtained from an individual who is negative for HLA-A*0201. It is generally preferred that the donor individual is otherwise HLA-identical to the patient. Antigen presenting cells (APCs) are then provided which display MHC molecules of the HLA-A*0201 allele and which are loaded with the peptide. The T cells of the donor individual are then primed with the APCs and the resulting cells are allowed to proliferate.

The proliferated T cells which are capable of binding the peptides used herein when in complex with the HLA-A*0201 antigen are then enriched using artificial structures which comprise a plurality of peptide-MHC molecules (e.g. pentamers or tetramers). The T cells specific for the particular peptide-HLA-A*0201 complex within the mixture of T cells have the capacity to bind to these structures when mixed with them. The T cells are subsequently mixed with magnetic beads with the capacity to bind the artificial structures. The artificial structures and the T cells bound to them are then removed from the remainder of the mixture by magnetic attraction of the beads.

Therapy

Antigen-specific T cells can be administered to an animal as frequently as several times daily or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An antigen specific T cell may be co-administered with the various other compounds (cytokines, chemotherapeutic and/or antiviral drugs, among many others). Alternatively, the compound(s) may be administered an hour, a day, a week, a month, or even more, in advance of an antigen specific T cell, or any permutation thereof. Further, the compound(s) may be administered an hour, a day, a week, or even more, after administration of an antigen specific T cell, or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the antigen specific T cell, and the like.

In the method of treatment, the administration of an antigen-specific T cell composition may be for either “prophylactic” or “therapeutic” purpose. When provided prophylactically, the composition is provided in advance of any symptom, although in particular embodiments a vaccine is provided following the onset of one or more symptoms to prevent further symptoms from developing or to prevent present symptoms from becoming worse. The prophylactic administration of composition serves to prevent or ameliorate any subsequent infection or disease. When provided therapeutically, the pharmaceutical composition is provided at or after the onset of a symptom of infection or disease. Thus, the present T cell compositions may be provided either prior to the anticipated exposure to a disease-causing agent or disease state or after the initiation of the infection or disease.

An effective amount of the composition would be the amount that achieves this selected result of enhancing the immune response, and such an amount could be determined as a matter of routine by a person skilled in the art. For example, an effective amount of for treating an immune system deficiency against cancer or pathogen could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present embodiments without necessitating undue experimentation.

### EXAMPLES

The preferred embodiments are further described in detail by reference to the following experimental examples. These examples are provided for purposes of illustration only, and are not intended to be limiting unless otherwise specified. Thus, the preferred embodiments should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present embodiments and practice the claimed methods. The following working examples therefore, specifically point out the preferred embodiments, and are not to be construed as limiting in any way the remainder of the disclosure.

### Example 1: Restoration of Anti-HER2 CD4⁺ Th1 Responses in DC1 Vaccinated Breast Cancer Patients

The following studies were designed to explore the role of adjuvant type 1-polarized dendritic cell (“DC1”) vaccination.

DC1 Vaccination of HER2⁺ IBC Patients with Residual Disease Following Neoadjuvant Therapy

Four HER2⁺ IBC patients with residual disease following neoadjuvant therapy received adjuvant HER2-pulsed DC1 vaccines. Th11 immune responses of each patient were determined pre-DC1 vaccination, 3-months post-DC vaccination, and 6-months post-vaccination and were generated from patient PBMCs pulsed with six HER2 Class II peptides (SEQ ID NOS: 1-6) by measuring IFNγ production via ELISPOT as described above. Autologous DC1 vaccines were prepared as described previously. Responses were evaluated on: (1) the overall anti-HER2 responsivity (responding to ≥1 peptide), (2) the number of reactive peptides (response repertoire), and (3) the cumulative response across the six HER2 peptides.

Results:

Responsivity: Pre-vaccination, only one IBC patient produced an immune response, defined as >20 SFC/10⁶ cells in an experimental well after subtracting unstimulated background. Compared with pre-vaccination results, all vaccinated IBC patients produced an immune response, defined as >2-fold increase in anti-HER2 IFN-γ^{pos} Th1 responses.

Response Repertoire: FIG. 1 shows mean repertoire increased from 0.5±1 peptides pre-vaccination to 3.25±0.96 peptides at 3 months (p=0.01) and 4±0.8 peptides at 6 months (p=0.01).

Cumulative response: FIG. 2 shows mean cumulative response improved from 36.5±38.3 SFC/10⁶ pre-vaccination to 151.0±60.0 SFC/10⁶ at 3 months (p=0.04) and 198.4±39.7 SFC/10⁶ at 6 months (p=0.02).

Conclusions:

HER2-pulsed DC1 vaccination of HER2⁺ IBC patients with residual disease following treatment with neoadjuvant chemotherapy boosts anti-HER2 Th1 immune responses.

The anti-HER2 Th1 immune responses increase in both breadth (response repertoire) and depth (cumulative response).

### Example 2—In Vitro Expansion of HER2-Specific Th1 Cells

The following studies were designed to explore the role of adoptive T-cell transfer in restoring anti-HER2 Th1 immunity. T cells are expanded to a level necessary for adoptive therapy and epitope mapping studies while maintaining antigen specificity and cellular function.

Briefly, in vitro, HER2-specific Th1 cells were generated by co-culture with HER2-peptide pulsed DC1s and expanded using IL-2 alone or IL-2, IL-7, and IL-15. Th1 cells were subsequently expanded either by repeat HER2-peptide pulsed DC1 co-culture or via anti-CD3/CD28 stimulation. Fold expansion was defined as: (#T-cells post expansion/#T-cells pre expansion); specificity was measured by antigen specific IFNγ production by ELISA.

As will be shown herein, repeated co-culture of CD4⁺ T cells with HER2 peptide pulsed DC1s stimulated with IL-2, IL-7, and IL-15 results in a significant expansion of highly specific anti-HER2 Th1 cells, providing a potential population of cells for adoptive transfer. Co-culture with specific peptide specific DC1s and IL-2, IL-7, and IL-15 stimulation may mimic the lymph node environment and may be used to significantly expand any population of antigen specific Th1 cells.

Without wishing to be bound by any particular theory, it is believed that when a subject is vaccinated against a protein antigen (for example, a tumor target antigen), blood can be removed from the subject after vaccination and collected. The collected blood contains dendritic cell precursors as well as low levels of T cells specific for the tumor target antigen. DC precursors and T cells are separated from each other. DC precursors can be loaded/pulsed with tumor target protein/antigen and then activated to DC1 status. The antigen-specific DC1s can then be co-cultured with the T cells, and cytokines (IL-15, IL-7 and IL-2) are added to the co-culture in appropriate sequence. This cycle can be repeated weekly until T cells grow to sufficient numbers (e.g. 1×10⁹). The T cells can then be supplied to the original subject, infusing them with a large quantity of T cells that their body could not produce naturally. This large army of antigen-specific T cells can have strong anti-tumor activity.

### Example 3-Method for Expanding T Cells

HER2 Specific DC1 Preparation:

DC precursors were obtained from HER2 breast cancer patients (DCIS) who were vaccinated with HER2 peptide-pulsed DC1 vaccines, as described previously. DC precursors were obtained via tandem leukapheresis/countercurrent centrifugal elutriation. DCs were incubated at 3×10⁶ cells in 1 ml Macrophage Serum-free Medium (SFM-Gibco Life Technologies, Carlsbad, Calif.) with GM-CSF 50 ng/ml (Berlex, Richmond, Calif.) at 37° C. DCs were pulsed with a single HER2 peptide antigen (42-56, 98-114, 328-345, 776-790, 927-941, 1166-1180 (SEQ ID NOS 1-6)); 20 μg/ml) 48-72 hrs after the cells were initially plated. For maturation, DCs were further activated 6 hours later with IFN-γ (1000 U/ml) and the following day with lipopolysaccharide (“LPS”) (10 ng/ml). HER2 specific DC1s were harvested 6 hours after LPS administration at the point of maximum IL-12 production.

CD4⁺ T Cell Preparation:

Lymphocytes were also obtained from previously vaccinated (HER2-pulsed DC1 vaccination) breast cancer patients via tandem leukapheresis/countercurrent centrifugal elutriation. CD4⁺ T-cells were purified by negative selection using Human CD4+ T Cell Enrichment Kit (Stemcell Technologies; Vancouver BC, Canada). CD4+ T-cells were resuspended at 2×10⁶ cells/ml in culture medium (ISOCOVE's Medium, 1% L-Glutamine, 1% Pen/Strep, 1% Sodium Pyruvate, 1% non-essential amino acids, Mediatech; Manassas, Va. and 5% heat inactivated human AB serum)

DC1-CD4⁺ Co-Culture:

DC1s were plated with CD4⁺ T-cells at a 1:10 ratio (2×10⁵ DC1s/ml with 2×10⁶ CD4⁺ T-cells/ml) in 24-well plates and incubated at 37° C. Recombinant Human IL-7 (10 ng/ml) and IL-15 (10 ng/ml) (BioLegend; San Diego, Calif.) were added 48-72 hrs after co-culture. Twenty-four hours after adding IL-7 and IL-15, Recombinant Human IL-2 (5 U/ml) was added.

HER2 Specific iDCs for Testing and HER2 Specific DC1s for Restimulation:

Two additional groups of DCs were prepared as described above. In one group, each well was pulsed with a single peptide antigen (20 ug/ml), and was considered as immature DCs (“iDCs”). In the second group, each well was pulsed with a single peptide antigen (20 μg/ml) and matured to DC1s as described above. Seven to nine days following the previous DC1 co-culture, the HER2 specific CD4⁺ T-cells were harvested. The T-cells were co-cultured with iDCs for ELISA testing. Interferon gamma production was measured by ELISA assay according to manufacturer's recommendations and protocols. The T-cells were also co-cultured with DC1s and stimulated with IL-7/15 and IL-2 as described above. The cycle was repeated with co-culture of CD4⁺ T-cells with HER2 specific DC1s a total of 4 times.

Expansion Method Outline:

Develop HER2-specific DC1s as previously described

Day 1: Culture monocytes (1 well of each peptide)

Day 4: Mature HER2-specific DC1s
AM: pulse with antigen (20 μg/ml).
PM (6 hrs later): add IFN-γ

Day 5: Purify CD4⁺ T-cells & DC1 co-culture.
AM: LPS
6 hrs later: Co-Culture: 2×10⁶ CD4⁺ T cells with 2×10⁵ DC1s
   *48-72 hrs after co-culture: add IL-7 (10 ng/ml) and IL-15 (10 ng/ml)
   24 hrs after adding IL-7/15: add IL-2 (5 U/ml)
   (In the IL-2 alone condition, IL2 was added 72-96 hrs after co-culture (at the same time as IL-2 was added to the IL2/7/15 group)
   Develop BOTH HER2-specific iDCs for ELISA testing as well as HER2-specific DC1s for restimulation

Day 9-11: Culture monocytes (2 wells of each peptide)

48 hrs after culturing monocytes: process HER2-specific immature DCs and mature DC1s
iDCs: Pulse with antigen (20 μg/ml) (given at the same time to future iDCs and DC1s)
DC1s: AM: pulse with antigen (20 μg/ml).
   PM (6 hrs later): add IFN-g

The next day: iDC co-culture & DC1 co-culture (i.e.: 7-9 days following DC1 co-culture)
DC1s: AM: LPS
Harvest HER2 specific CD4⁺ Tcells
Harvest HER2 specific iDCs
   Co-Culture: 2×10⁶ CD4⁺ Tcells with 2×10⁵ iDCs
Harvest HER2 specific DC1s
   Co-Culture: 2×10⁶ CD4⁺ Tcells with 2×10⁵ DC1s

The next day: Run ELISA on iDC co-culture

Repeat process above, resume at IL7/15 stimulation (*)

### Results:

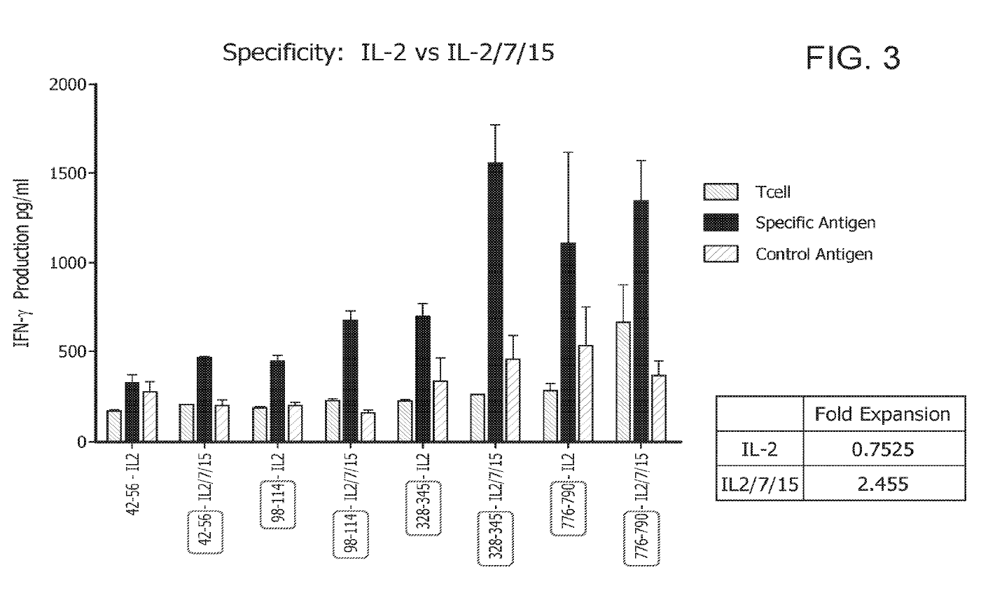

The above-referenced methods were used in the experiments and studies for which the results are shown in FIGS. 3-16:

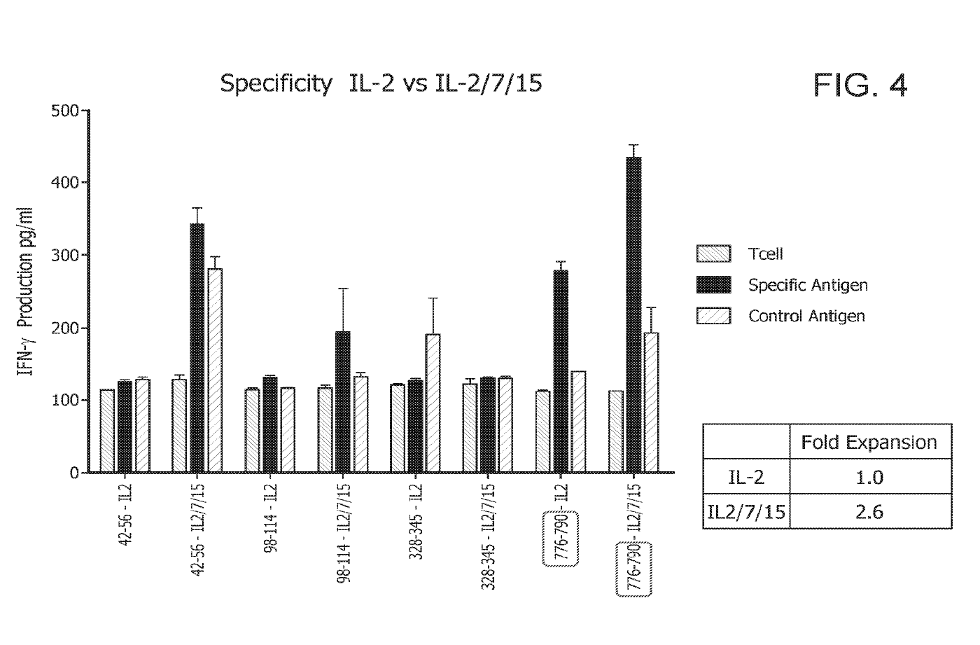

FIG. 3 and FIG. 4 show a direct comparison between CD4⁺ T cells co-cultured with HER2-specific DC1's stimulated with IL-2 versus those stimulated with IL-2/7/15 for two different patients who had received HER2-pulsed DC1 vaccination, respectively.

Briefly, immature DC's (“iDC's”) from the respective patients were pulsed with the following MHC class II peptides: peptide 42-56 (SEQ ID NO: 1), peptide 98-114 (SEQ ID NO: 2), peptide 328-345 (SEQ ID NO: 3), and peptide 776-790 (SEQ ID NO: 4) and matured to DC1's. The resulting HER2-pulsed DC1's were then co-cultured with CD4⁺ T cells and stimulated with IL-2 alone or with IL-2/7/15 as indicated. The red outline boxes indicate the specific peptide and stimulation protocol for which specificity is shown (greater than 2:1 ratio of specific antigen:control antigen IFN-γ production). Thus in FIG. 3 specificity was shown for peptide 42-56-IL2/7/15 protocol; peptide 98-114-IL-2 protocol and IL-2/7/15 protocol, peptide 328-345 both protocols, and peptide 776-790, both protocols. In FIG. 4 specificity was shown for peptide 776-790, both protocols, only. Graphs showing fold expansion (defined as number of T cells post expansion/number of T cells pre expansion) are shown at right in each figure. In general, fold expansion was greater for the IL-2/7/15 stimulation than for IL-2 stimulation alone as shown by the respective fold expansion data. Specificity was measured by antigen-specific IFN-γ production by ELISA.

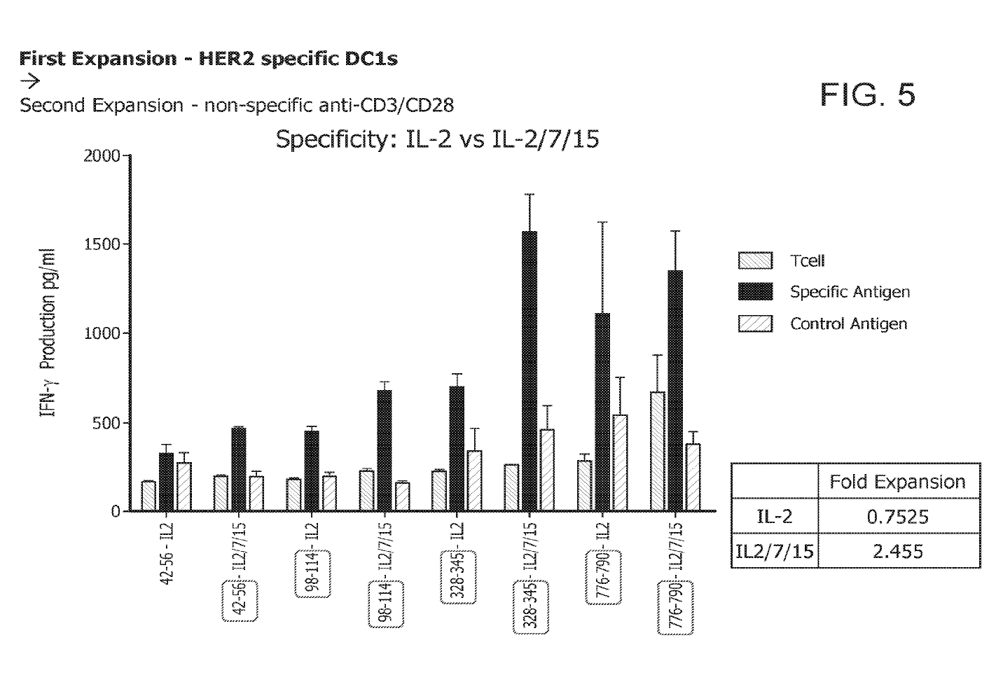

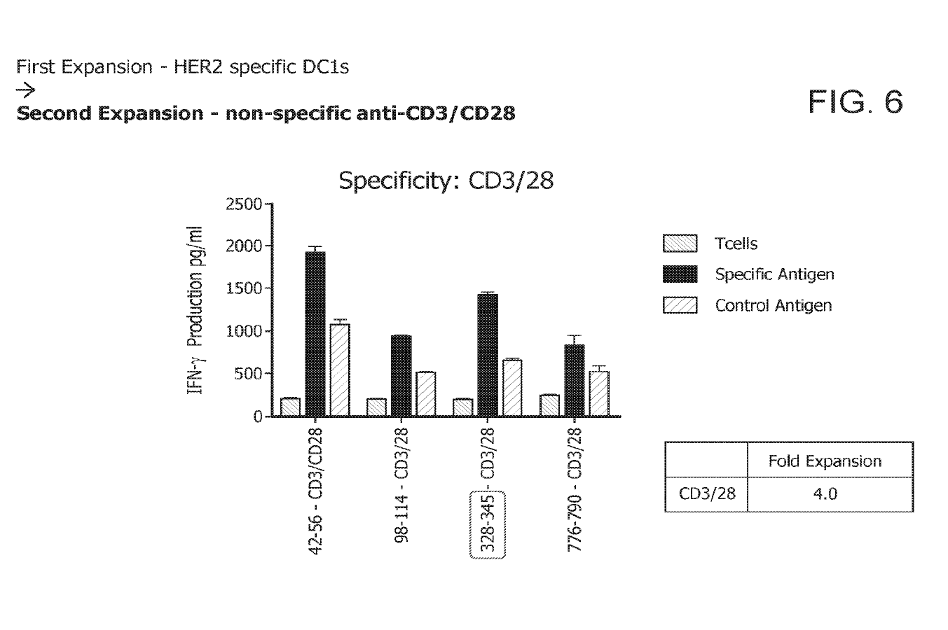

FIG. 5 and FIG. 6 show specific responses followed by non-specific immune responses: FIG. 5 shows a specific response following a first stimulation/expansion with HER2-specific DC1's and FIG. 6 shows the subsequent loss of that specific response after the second stimulation/expansion with non-specific anti CD3/CD28.

The first stimulation of CD4; T cells with HER2-specific DC1s resulted in multiple specific immune responses as shown by red outline boxes in FIG. 5: peptide 42-56, IL2/7/15 protocol; peptide 98-114, both protocols, peptide 328-345, both protocols, and peptide 776-790, both protocols. FIG. 6 shows the second stimulation of the HER2-specific CD4⁺ T cells with a non-specific anti-CD3/CD28 stimulus resulted in a four-fold expansion (side graph), but with a loss of specificity in three fourths of the peptide groups (only peptide 328-345 showed specificity after CD3/28 expansion). iDC's from patients were pulsed with the following MHC class II peptides: peptide 42-56 (SEQ ID NO: 1), peptide 98-114 (SEQ ID NO: 2), peptide 328-345 (SEQ ID NO: 3), and peptide 776-790 (SEQ ID NO: 4) and matured to DC1's as described above. The resulting HER2-pulsed DC1's were then co-cultured with CD4⁺ T cells and stimulated with IL-2 alone or with IL-2/7/15 as indicated.

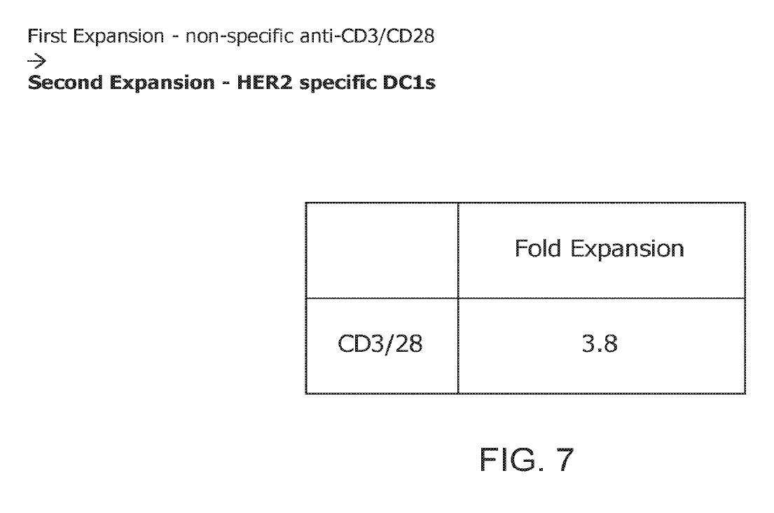

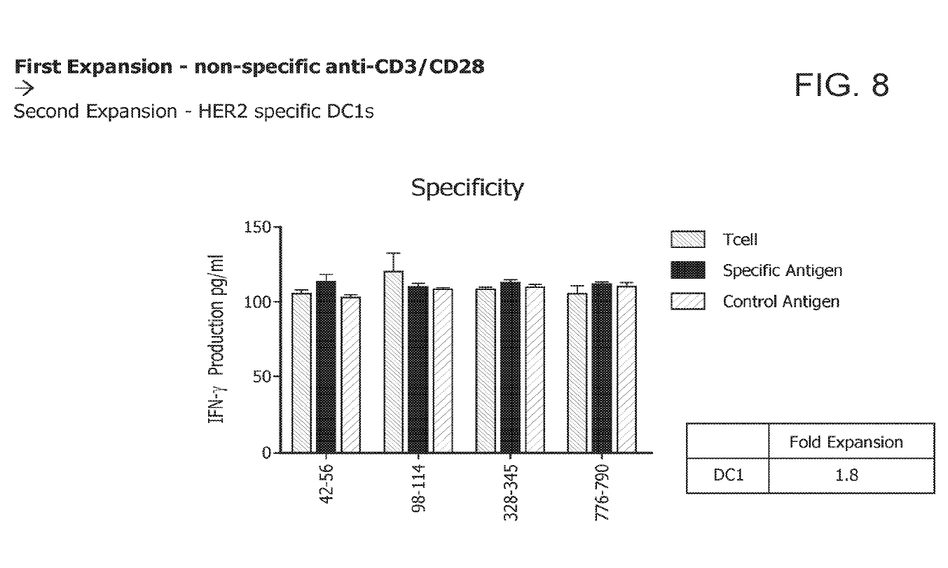

FIG. 7 and FIG. 8 show non-specific immune response followed by specific immune responses: FIG. 7 shows non-specific expansion of CD4⁺ T cells. FIG. 8 shows failure to obtain specificity following subsequent stimulation with HER2-specific DC1's. The first stimulation of CD4⁺ T cells with non-specific anti CD3/CD28 resulted in a 3.8 fold expansion (FIG. 7). The second stimulation of the non-specific CD4⁺ T cells with HER2-specific DC1's failed to result in a specific immune response (FIG. 8). iDC's from patients were pulsed with the following MHC class II peptides: peptide 42-56 (SEQ ID NO: 1), peptide 98-114 (SEQ ID NO: 2), peptide 328-345 (SEQ ID NO: 3), and peptide 776-790 (SEQ ID NO: 4) and matured to DC1's as described above. The resulting HER2-pulsed DC1's were then co-cultured with CD4⁺ T cells and stimulated with IL-2 alone or with IL-2/7/15 as indicated.

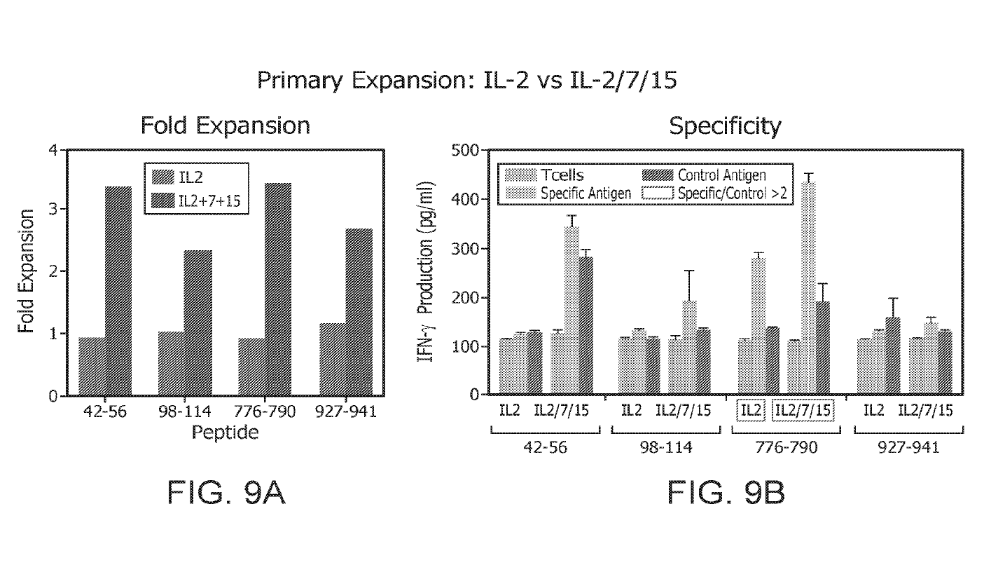

FIGS. 9A and 9B show in vitro primary/first expansion of HER2-specific Th1 cells comparing CD4⁺ T cells co-cultured with HER2-specific DC1's expanded with IL-2 versus those expanded with IL-2/7/15. iDC's were pulsed with the following MHC class II peptides: peptide 42-56 (SEQ ID NO: 1), peptide 98-114 (SEQ ID NO: 2), peptide 776-790 (SEQ ID NO: 4) and peptide 927-941 (SEQ ID NO: 5), and) and matured to DC1's. The resulting HER2-pulsed DC1's were then co-cultured with CD4⁺ T cells and stimulated with IL-2 alone or with IL-2/7/15 as indicated. The red outline boxes (FIG. 9B) indicate the specific peptide and stimulation protocol for which specificity is shown (greater than 2:1 ratio of specific antigen:control antigen IFN-γ production). FIG. 9A shows mean fold expansion (defined as number of T cells post-expansion/number of T cells pre-expansion) of Th1 cells was significantly better when stimulated with IL-2, IL-7, and IL-15 than with IL-2 alone (2.6±0.75 vs 1.0±0.12; p=0.001). FIG. 9B shows specificity for the various peptide/expansion protocols as measured by antigen-specific IFN-γ production by ELISA. Both stimulation with IL-2, IL-7, and IL-15 and with IL-2 alone resulted in a specific Th1 response in the same HER2 peptide 776-790.

Primary expansion summary: IL-2 vs. 1-2/7/15. Mean fold expansion of Th1 cells was significantly better when stimulated with IL-2, IL-7, and IL-15 than with IL-2 alone (2.6±0.75 vs 1.0±0.12; p=0.001) (FIG. 9A). Both stimulation with IL-2, IL-7, and IL-15 and with IL-2 alone resulted in a specific Th1 response in the same HER2 peptide (peptide 776-790) (FIG. 9B).

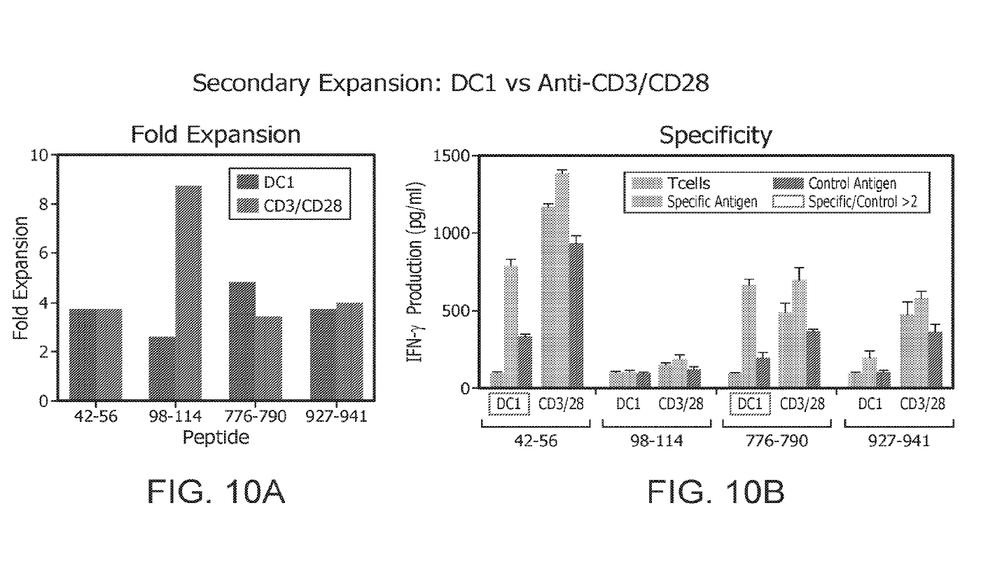

FIGS. 10A and 10B show in vitro secondary/second expansion of HER2-pulsed DC1's versus anti-CD3/CD28. Re-stimulation of Th1 cells with HER2-peptide pulsed DC1s and anti-CD3/CD28 each resulted in a similar fold expansion (3.9±1.0 vs. 4.3±2.0 p=0.7) (FIG. 10A). However, FIG. 10B shows stimulation of the Th1 cells with HER2-specific DC1s enhanced the specific Th1 response; whereas non-specific stimulation with anti-CD3/CD28 resulted in an overall loss of HER2-peptide specificity. The following MHC class II peptides were used: peptide 42-56 (SEQ ID NO: 1), peptide 98-114 (SEQ ID NO: 2), peptide 776-790 (SEQ ID NO: 4) and peptide 927-941 (SEQ ID NO: 5). The red outline boxes (FIG. 10B) indicate the specific peptide and stimulation protocol for which specificity is shown (greater than 2:1 ratio of specific antigen:control antigen IFN-γ production) (i.e., DC1 restimulation of peptide 42-56-specific Th1 Cells and peptide 776-790-specific Th1 cells.

Secondary expansion summary: HER2-peptide pulsed DC1 vs. anti-CD3/CD28. Re-stimulation of Th1 cells with HER2-peptide pulsed DC1s and anti-CD3/CD28 each resulted in a similar fold expansion (3.9±1.0 vs. 4.3±2.0, p=0.7) (FIG. 10A). However, stimulation of the Th1 cells with HER2-specific DC1s enhanced the specific Th1 response, whereas non-specific stimulation with anti-CD3/CD28 resulted in an overall loss of HER2-peptide specificity (FIG. 10B).

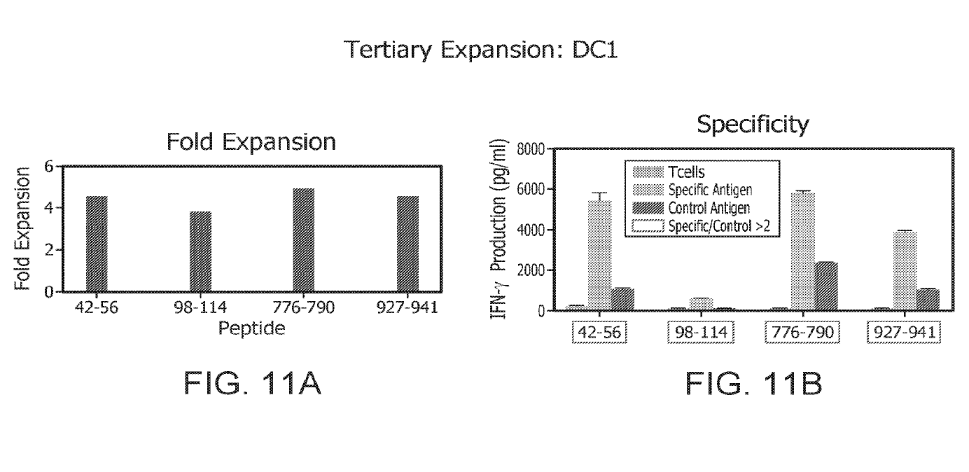

Tertiary expansion of the Th1 cells with HER2-pulsed DC1's Following a third stimulation with indicated HER2-specific DC1s, both mean fold expansion (4.32±0.5, 43.7-fold cumulative expansion (FIG. 11A) and antigen specificity (FIG. 11B) increased again, specifically all four peptides (peptide 42-56 (SEQ ID NO: 1), peptide 98-114 (SEQ ID NO: 2), peptide 776-790 (SEQ ID NO: 4) and peptide 927-941 (SEQ ID NO: 5)) show specificity and increased IFN-γ production.

Tertiary expansion summary: HER2-peptide pulsed DC1. Following a third stimulation with HER2-specific DC1s, both mean fold expansion (4.32±0.5, 43.7-fold cumulative expansion) (FIG. 11A) and specificity-both the number of specific peptides and IFN-γ production (FIG. 11B) increased again.

Overall it is seen In FIGS. 9B, 10B, and 11B, which have the same numbers of T cells, the IFN-γ production goes up by logs from expansion to expansion. It was also seen that in the second stimulation, non-specific CD3/CD28 (FIG. 10B), there was an overall loss of specificity. The cells were expanded as Th1 phenotype with 50-200-fold expansion (FIGS. 9A, 10A, and 11A) that became more specific and stronger with each stimulation.

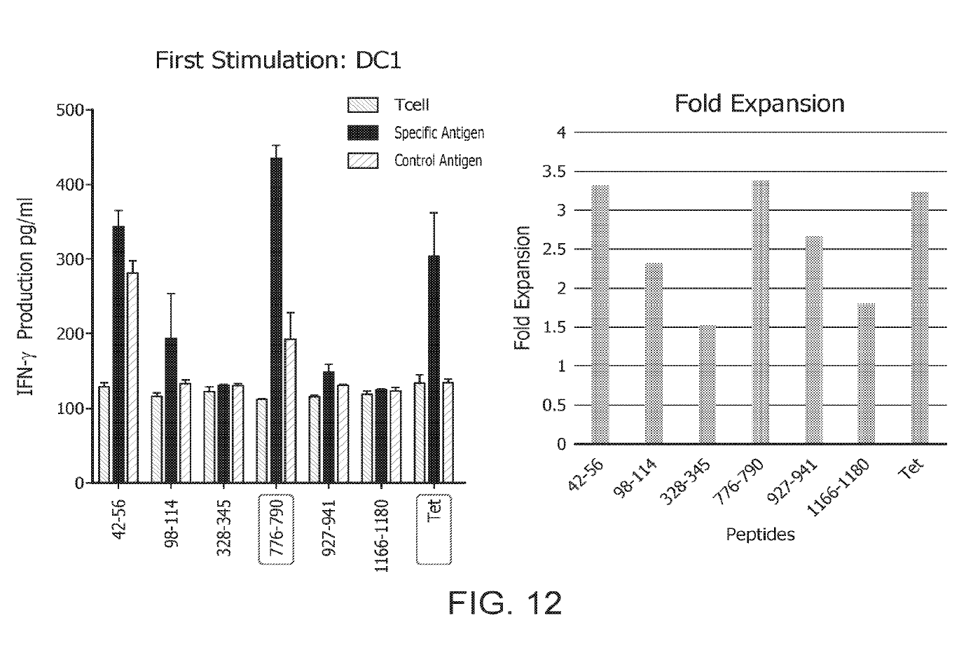

FIGS. 12-15 show sequential results of repeated in vitro stimulation (4 times) of HER2-specific CD4⁺ Th1 cells with IL-2/7/15. For all results shown in FIGS. 12-15 the respective left panels show peptide specificity by IFN-γ production (“Tet” is a tetanus patient control); respective right panels show fold expansion for the specific HER2-peptides used. In FIG. 12 an additional MHC-class II peptide was used to pulse iDC's: peptide 1166-1180 (SEQ ID NO: 6) in addition to the other five used in the above studies. However, as seen in the fold expansion results (FIG. 12, right panel), peptide 328-345-specific Th1 cells and peptide 1166-1180-specific Th1 cells did not produce enough cells for further expansion thus only HER2 Th1 cells specific to the remaining four peptides were used.

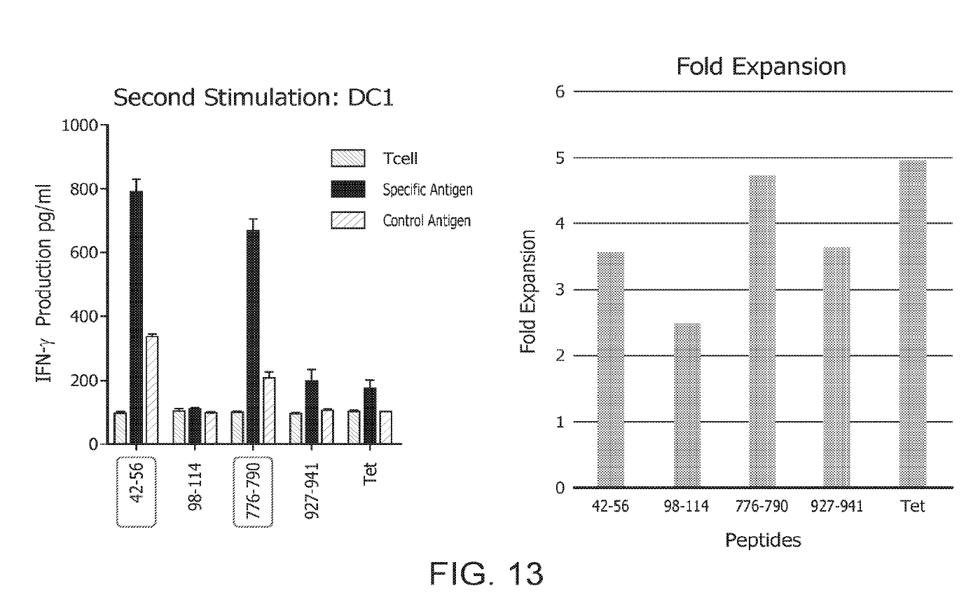

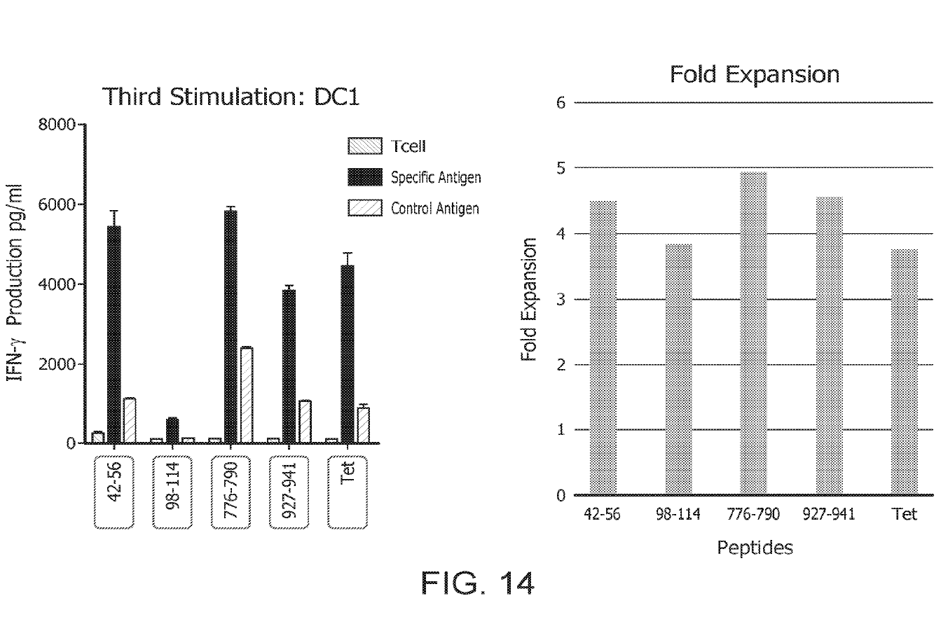

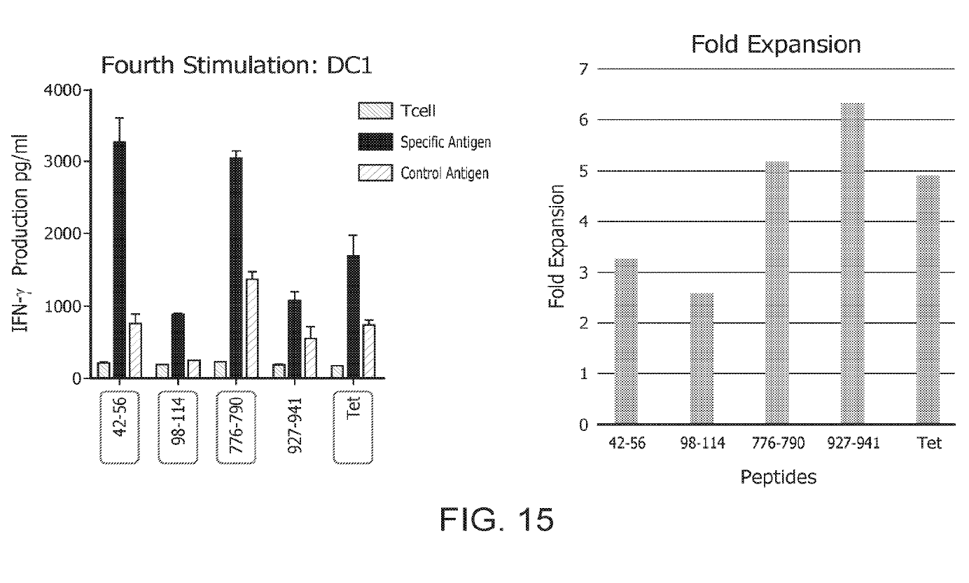

Sequentially, FIG. 12 for the first stimulation shows specificity only for peptide 776-790-specific Th1 cells; FIG. 13 for the second stimulation shows an increase, specificity for peptide 42-56 in addition to peptide 776-790-specific Th1 cells; FIG. 14 for the third expansion shows specificity for all four peptide-specific Th1 cells (peptide 42-56, peptide 98-114, peptide 776-790, and peptide 927-941), and FIG. 15 for the fourth expansion shows loss of specificity for one of the peptides (peptide 927-941) leaving three remaining HER2-specific peptides (peptide 42-56, peptide 98-114, and peptide 776-790).

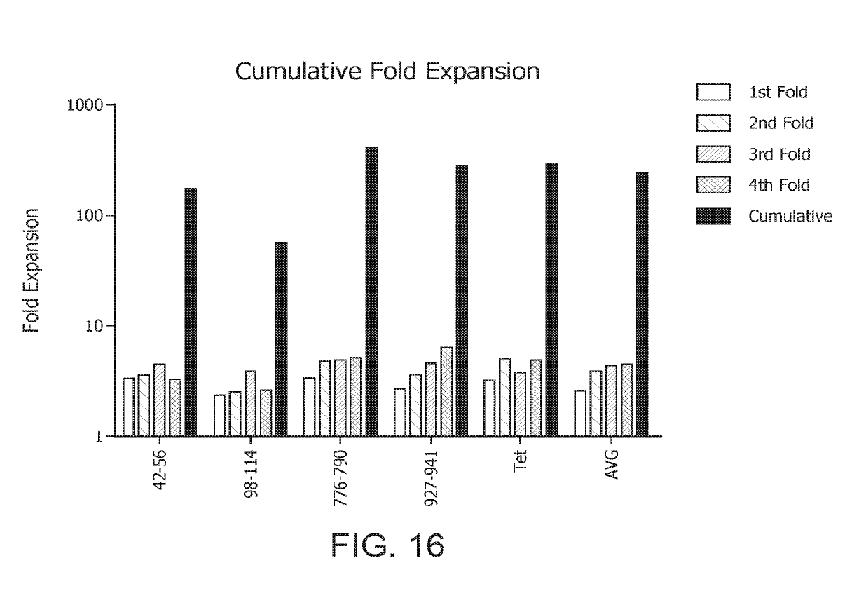

FIG. 16 shows cumulative fold expansion of the four expansions shown in FIGS. 12-15 for all the HER2-specific Th1 cells, with the last bar of each group (dots) showing cumulative fold expansion. Average cumulative fold expansion was over 100-fold.

Conclusions:

Repeated co-culture of CD4⁺ T cells with HER2-peptide pulsed DC1s stimulated with IL-2, IL-7, and IL-15 results in a significant expansion of highly specific anti HER2 Th1 cells, providing a potential population of cells for adoptive transfer.

Each stimulation out of the total four stimulations resulted in both increased fold expansion and increased antigen specificity, without reaching a limit of either. Indeed there was shown a 100-400-fold expansion.

Co-culture with peptide specific DC1s and IL-2, IL-7, and IL-15 stimulation may mimic the lymph node environment and be used to significantly expand any population of antigen specific Th1 cells. Those skilled in the art will readily recognize that the present embodiments related to T cell expansion are in no way limited to CD4⁺ t cells. Thus the present embodiments provide methods for growing CART cells, cytotoxic T lymphocytes (CD8⁺'s) as well as all other kinds of T cells.

The results presented herein demonstrate that HER2-pulsed DC1 vaccination of HER2⁺ IBC patients with residual disease following treatment with neoadjuvant chemotherapy boosts anti-HER2 Th1 immune responses. The anti-HER2 Th1 immune response increases in both breadth (response repertoire) and depth (cumulative response). Adoptive transfer of HER2-specific Th1 cells may serve a role in resurrecting the CD4+ Th1 immune response. Repeated co-culture with HER2-peptide pulsed DC1s stimulated with IL-2, IL-7, and IL-15 can result in significant expansion of highly specific anti-HER2 Th1 cells.

The disclosures of each and every patent, patent application, and publication cited herein are hereby incorporated herein by reference in their entirety.

This disclosure has been presented for purposes of illustration and description but is not intended to be exhausting or limiting. Many modifications and variations will be apparent to those of ordinary skill in the art. The embodiments were chosen and described in order to explain principles and practical application, and to enable others of ordinary skill in the art to understand the disclosure for various embodiments with various modifications as are suited to the particular use contemplated.

Although illustrative embodiments have been described herein with reference to the accompanying drawings, it is to be understood that the embodiments are not limited to those particular descriptions, and that various other changes and modifications may be devised therein by one skilled in the art without departing for the scope or spirit of the disclosure. The appended claims are intended to be construed to include all such embodiments and equivalent variations.

What is claimed is:

1. A method of expanding a T cell population which comprises at least one T cell obtained from a blood sample from a subject who has been vaccinated against an antigen, comprising the step of: contacting said T cell with one or more of a dendritic cell (“DC”) or a precursor thereof, at least two cytokines, and a T cell growth factor.

2. The method of claim 1, wherein said blood sample contains at least one T cell of said population specific for said vaccine antigen and at least one DC precursor.

3. The method of claim 1, wherein said DC precursor is pulsed with said antigen and activated to an antigen-specific type I dendritic cell (“DC1”) and then co-cultured with said T cell to generate an antigen-specific DC1.

4. The method of claim 1, wherein the at least two cytokines comprises interleukin-7 (“IL-7”) and interleukin-15 (“IL-15”).

5. The method of claim 1, wherein said T cell growth factor comprises interleukin-2 (“IL-2”).

6. The method of claim 1, further comprising the steps of:
a) co-culturing said T cell from said patient sample with said antigen-specific T cell autologous type I dendritic cell (DC1) in vitro;
b) contacting said cell from step a) with IL-7 and IL-5 to generate a stimulated antigen-specific T cell; and
c) subsequently contacting said stimulated antigen specific T cell with IL-2, thereby generating an expanded antigen specific T cell population that maintains antigen specificity and cellular function.

7. The method of claim 6, further comprising repeating steps a) through c) from one to at least three additional times to generate further expanded antigen-specific T cell populations.

8. The method of claim 1 wherein said T cell is CD4⁺.

9. The method of claim 1 wherein said antigen is HER2.

10. A method of expanding a CD4⁺ T cell population which comprises at least one CD4⁺ T cell obtained from a blood sample from a breast cancer patient who has been vaccinated against HER2, comprising the step of: contacting said CD4⁺ T cell with one or more of a dendritic cell (“DC”) or a precursor thereof, at least two cytokines, and a T cell growth factor.

11. The method of claim 10, wherein at least one DC precursor in said sample is pulsed with at least one HER2 MHC class II peptide and is contacted with said CD4⁺ T cell.

12. The method of claim 10, wherein the at least two cytokines comprises interleukin-7 (“IL-7”) and interleukin-15 (“IL-15”).

13. The method of claim 10, wherein said T cell growth factor comprises interleukin-2 (“IL-2”).

14. The method of claim 10, comprising:
a) co-culturing said T cell from claim 11 with said HER2-pulsed DC1;
b) contacting the cell from step a) with IL-7 and IL-15 to generate a stimulated antigen-specific T cell; and
c) subsequently contacting said stimulated antigen specific T cell with IL-2, thereby generating an expanded antigen specific T cell population that maintains antigen specificity and cellular function.

15. The method of claim 14, further comprising repeating steps a) through c) from one to at least three additional times to generate further expanded antigen-specific T cell populations.

16. The method of claim 10, wherein said sample is pulsed with HER2 MHC class II peptides, comprising:
Peptide 42-56:
(SEQ ID NO: 1)
HLDMLRHLYQGCQVV;
Peptide 98-114:
(SEQ ID NO: 2)
RLRIVRGTQLFEDNYAL;
Peptide 328-345:
(SEQ ID NO: 3)
TQRCEKCSKPCARVCYGL;
Peptide 776-790:
(SEQ ID NO: 4)
GVGSPYVSRLLGICL;
Peptide 927-941:
(SEQ ID NO: 5)
PAREIPDLLEKGERL;
and
Peptide 1166-1180:
(SEQ ID NO: 6)
TLERPKTLSPGKNGV.
